(19) United States
(12) Patent Application Publication
(10) Pub. No.: US 2024/0166990 A1
(43) Pub. Date: May 23, 2024

(54) METHODS AND COMPOSITIONS FOR INCREASING PROTEIN EXPRESSION IN GENETICALLY ENGINEERED BACTERIA

(71) Applicant: Azitra Inc., Branford, CT (US)

(72) Inventors: Roger LEGER, Montreal (CA); Dorival MARTINS, Montreal (CA)

(21) Appl. No.: 18/362,205

(22) Filed: Jul. 31, 2023

Related U.S. Application Data
(63) Continuation of application No. PCT/US2022/014741, filed on Feb. 1, 2022.
(60) Provisional application No. 63/144,076, filed on Feb. 1, 2021.

(51) Int. Cl. C12N 1/20; A61K 38/55; A61P 31/04; C12N 1/36

(52) U.S. Cl. CPC C12N 1/205; A61K 38/55; A61P 31/04; C12N 1/36; C12N 2500/34; C12N 2500/36; C12N 2500/38

(57) ABSTRACT

The present disclosure provides, inter alia, a composition and method to make the composition comprising a therapeutic protein expressing microorganism, one or more disaccharides, one or more oligosaccharides, one or more antioxidants, one or more fatty alcohol, and colloidal oatmeal and/or alkaline oatmeal extract.

### RELATED APPLICATIONS

This application is a Continuation of International Application Serial No. PCT/US2022/014741, filed on Feb. 1, 2022, which claims priority of U.S. Provisional Application No. 63/144,076 filed Feb. 1, 2021, the contents of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 31, 2023, is named 129062-02002.xml and is 72,497 bytes in size.

### FIELD OF THE DISCLOSURE

The present disclosure relates to methods, kits, and compositions for increasing protein expression in genetically engineered bacteria.

### BACKGROUND OF THE INVENTION

A recent increase in the application of using genetically engineered bacteria as a targeted delivery system of a protein or peptide has become attractive as therapeutic agents. This kind of genetically engineered bacteria is sometimes called recombinant probiotic bacteria (US 2018/0161380 A1, A. M. Munivar et al., US 2018/0135062 A1, T. Wirth et al., WO 2017/044836 A1, T. Hitchcock et al., WO 2017/147507 A1, T. Hitchcock et al.). Other examples include bacteria strains that produce a natural product with a therapeutic value (WO 2019/046801 A1, R. L. Gallo et al.).

The current state of formulations in this area involves either placing the lyophilized or freeze-dried bacteria in a capsule or a cream. The capsule will dissolve in the mucous membrane or fluid while a cream can be applied directly to the target area.

There remains a need to develop improved formulations of compositions comprising genetically engineered bacteria to increase protein expression and continuously express the protein of interest.

### SUMMARY OF THE INVENTION

According to one aspect, the present disclosure provides a composition comprising one or more viable microorganisms and a first mixture, wherein the one or more microorganism produces a therapeutic agent, wherein the first mixture comprises one or more disaccharides, one or more oligosaccharides, and one or more antioxidants, and wherein the composition has viability of at least 1×10¹⁰ CFU/g.

According to some embodiments, the composition further comprising one or more fatty alcohols or alcohol.

According to some embodiments, the one or more disaccharides is selected from the group consisting of lactose, trehalose, sucrose, maltose, and cellobiose.

According to some embodiments, the one or more oligosaccharides is selected from the group consisting of fructo-oligosaccharides (FOS), galactooligosaccharides (GOS), mannan oligosaccharides (MOS), isomalto-oligosaccharides, xylo-oligosaccharides, galacto-oligosaccharides, and raffinose.

According to some embodiments, the one or more antioxidants is selected from the group consisting of ascorbic acid, vitamin A, vitamin E, a-carotene, lycopene, lutein, zeaxanthin, and water soluble derivatives of lipophilic antioxidants.

According to some embodiments, the water soluble derivatives of lipophilic antioxidants are alpha-tocopherol phosphate or alpha-tocopherol polyethylene glycol ester.

According to some embodiments, the one or more disaccharides is lactose, the one or more oligosaccharides is fructo-oligosaccharides (FOS), and the one or more antioxidants is ascorbic acid.

According to some embodiments, the one or more fatty alcohols is selected from the group consisting of saturated fatty alcohol and unsaturated fatty alcohol.

According to some embodiments, the saturated fatty or alcohol acid is cetyl alcohol and the unsaturated fatty alcohol is oleyl alcohol. According to some embodiments, the mixture of oleyl alcohol and cetyl alcohol ranges from about 25% oleyl alcohol/75% cetyl alcohol to about 50% oleyl alcohol/50% cetyl alcohol (w/w).

According to some embodiments, the second mixture comprises oleyl alcohol and cetyl alcohol at about a 1:1 ratio.

According to some embodiments, the one or more disaccharides is about 50% (w/w) to about 55% (w/w), the one or more oligosaccharides is about 10% (w/w) to about 15% (w/w), the one or more antioxidants is about 0.1% (w/w) to about 2% (w/w), and the one or more microorganism is about 30% (w/w) to about 35% (w/w).

According to some embodiments, the composition of the microorganisms and the first mixture is about a 10:1 ratio with a second mixture comprising the one or more fatty alcohols.

According to some embodiments, the second mixture further comprises colloidal oatmeal and/or alkaline oatmeal extract. According to some embodiments, the second mixture comprises colloidal oatmeal at about 1% (wt/wt) and the alkaline oatmeal extract at about 0.5% (wt/wt) when in combination with the fatty alcohols.

According to some embodiments, the microorganism is selected from the group consisting of Bifidobacterium, Brevibacterium, Propionibacterium, Lactococcus, Streptococcus, Staphylococcus, Lactobacillus, Enterococcus, Pediococcus, Leuconostoc, Oenococcus, or Corynebacterium and mixtures thereof. According to some embodiments, the microorganism is Staphylococcus epidermidis.

According to some embodiments, the therapeutic agent is produced naturally by the microorganism. According to some embodiments, the microorganism is genetically engineered to produce the therapeutic agent.

According to some embodiments, the therapeutic agent is selected from a polypeptide, a small molecule, and a metabolite.

According to some embodiments, the therapeutic agent is selected from the group consisting of one or more LEKTI protein domains, filaggrins, interferons, enkephalins, interleukins, and antimicrobial agents. According to some embodiments, the one or more LEKTI protein domains is LEKTI-d6.

According to some embodiments, the one or more antimicrobial agents is selected from the group consisting of chitinase, a glucanase, or a peptidoglycan hydrolase.

According to some embodiments, the microorganism is attenuated by auxotrophy. According to some embodiments, the microorganism is a D-alanine auxotroph. According to some embodiments, the microorganism comprises a deletion of one or more of alr1, alr2, and dat genes.

According to some embodiments, the composition is administered to a mammal's skin.

According to some embodiments, the expression of the therapeutic agent is increased when compared to a composition without the second mixture.

According to some embodiments, the expression of the therapeutic agent is at least 2-fold, at least 3-fold, at least 4-fold, at least 5-fold, at least 6-fold, at least 7-fold, at least 8-fold, at least 9-fold, at least 10-fold, at least 11-fold, at least 12-fold, or at least 13-fold when compared to a composition without the second mixture after 8 hours. According to some embodiments, the expression of the therapeutic agent is at least 5-fold when compared to a composition without the second mixture after 8 hours. According to some embodiments, the expression of the therapeutic agent is at least 10-fold when compared to a composition without the second mixture after 8 hours.

According to some embodiments, the composition has viability of at least 2.5×10¹⁰ CFU/g, at least 5×10¹⁰ CFU/g, at least 1×10¹¹ CFU/g, at least 2.5×10¹¹ CFU/g, at least 5×10¹¹ CFU/g, at least 1×10¹² CFU/g, or at least 2.5×10¹² CFU/g. According to some embodiments, the composition has viability of about 1×10¹⁰ CFU/g to about 1×10¹¹ CFU/g, about 5×10¹⁰ CFU/g to about 5×10¹¹ CFU/g, about 1×10¹¹ CFU/g to about 1×10¹² CFU/g, or about 5×10¹¹ CFU/g to about 5×10¹² CFU/g.

According to some embodiments, the antimicrobial activity is at least 1.5-fold, at least 2-fold, at least 3-fold, at least 4-fold, or at least 5-fold when compared to a composition without the second mixture after 8 hours. According to some embodiments, the antimicrobial activity is at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, or at least 12-fold when compared to a composition without the second mixture after 24 hours.

According to some embodiments, the LEKTI protein domains activity is at least 1.5-fold, at least 2-fold, at least 3-fold, at least 4-fold, or at least 5-fold when compared to a composition without the second mixture after 8 hours. According to some embodiments, the LEKTI protein domains activity is at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, or at least 12-fold when compared to a composition without the second mixture after 24 hours.

According to another aspect, the present disclosure provides a pharmaceutical composition comprising any one of the compositions disclosed herein, and a pharmaceutically acceptable carrier. According to some embodiments, the pharmaceutically acceptable carrier is selected from the group consisting of an aqueous solution, an emulsion, a cream, a lotion, a gel, or an ointment

According to another aspect, the present disclosure provides a method for preparing any one of the compositions disclosed herein or any one of the pharmaceutical compositions disclosed herein, comprising: (a) combining the one or more microorganisms and the first mixture, wherein the first mixture comprises the one or more disaccharides, the one or more oligosaccharides, and the one or more antioxidants; (b) lyophilizing the resulting composition of step (a); and (c) combining the second mixture and the resulting lyophilized composition of step (b), wherein the second mixture comprises the one or more fatty alcohols.

According to another aspect, the present disclosure provides a method of treating a disease, disorder, or condition in a subject, the method comprising administering to the subject any one of the compositions disclosed herein or any one of the pharmaceutical compositions disclosed herein.

According to some embodiments, the disease, disorder, or condition is a skin disease or disorder, inflammatory disease, and cancer.

According to some embodiments, the skin disease or disorder is Netherton Syndrome, psoriasis, acne, atopic dermatitis, allergic contact dermatitis, epidermolytic hyperkeratosis, seborrheic dermatitis, eczema, dry skin, allergy, rashes, UV-irritated skin, detergent irritated skin, thinning skin, bullous pemphigoid, pemphigus vulgaris, impetigo, vitiligio, baldness, and/or hirsutism.

According to some embodiments, the disease or disorder is associated with pain and is selected from the group consisting of acute pain, chronic pain, nociceptive pain, neuropathic pain, traumatic pain, inflammatory pain, post-operative incision pain, pain associated with cancer, fracture pain, osteoporotic pain, bone cancer pain, and gout joint pain.

According to some embodiments, the cancer is selected from the group consisting of malignant melanoma, colon cancer, breast cancer, lung cancer, ovarian cancer, gastric cancer, oral tongue squamous cell carcinoma, squamous cell cancer, prostate cancer, pancreatic cancer, liver cancer, kidney cancer, bladder cancer, cervical cancer, endometrial cancer, gallbladder cancer, brain cancer and oral cancer.

According to another aspect, the present disclosure provides a method of increasing expression of a therapeutic agent by a microorganism, comprising preparing any one of the composition disclosed herein or any one of the pharmaceutical composition disclosed herein, comprising: (a) combining the one or more microorganisms and the first mixture, wherein the first mixture comprises the one or more disaccharides, the one or more oligosaccharides, and the one or more antioxidants; (b) lyophilizing the resulting composition of step (a); and (c) combining the second mixture and the resulting lyophilized composition of step (b), wherein the second mixture comprises the one or more fatty alcohols.

### BRIEF DESCRIPTION OF THE DRAWINGS

FIG. 1A-1H depicts graphs showing the effect of media formulation on SE27a growth (FIGS. 1A-1D) and LEKTI-d6 production (FIGS. 1E-1H) in liquid cultures. Growth of SE27a is displayed on FIG. 1A ASM media, FIG. 1B TSB media, FIG. 1C ASM+1% colloidal oatmeal (CO) and FIG. 1D ASM+0.5% colloidal oatmeal extract (COE). Active LEKTI production is displayed on FIG. 1E ASM media, FIG. 1F TSB media, FIG. 1G ASM+1% CO and FIG. 1H ASM+0.5% COE.

FIG. 2 depicts a graph showing the effect of addition of Colloidal Oatmeal (CO) and Colloidal Oatmeal Extract (COE) on bacterial growth on agar plates.

FIG. 3 depicts a graph showing the effect of addition of Colloidal Oatmeal (CO) and Colloidal Oatmeal Extract (COE) on active LEKTI levels on agar plates.

FIG. 4 depicts a graph showing growth of bacterial growth on ASM agar plates of SE-27 formulated in oleyl alcohol:cetyl alcohol (OA:CA), OA:CA+colloidal oatmeal (CO) and OA:CA+colloidal oatmeal extract (COE).

FIG. 5 depicts a graph showing active LEKTI-d6 production of SE-27a formulated in oleyl alcohol:cetyl alcohol (OA:CA), OA:CA+colloidal oatmeal (CO) and OA:CA+colloidal oatmeal extract (COE) after growth on ASM agar plates over 8 hours.

FIG. 6 depicts a graph showing the bacterial content of placebo or SE-27a formulated in oleyl alcohol:cetyl alcohol (OA:CA)+colloidal oatmeal (CO) over a 24-h incubation on a pig skin surface.

FIG. 7 depicts a graph showing active LEKTI-d6 levels of placebo or SE-27a formulated in oleyl alcohol:cetyl alcohol (OA:CA)+colloidal oatmeal (CO) over a 24-h incubation on a pig skin surface.

FIG. 8A depicts a graph showing growth of SE484 in artificial sweat media containing (ASM) 2% D-alanine in presence or absence of 1% Colloidal oatmeal.

FIG. 8B depicts a graph showing antimicrobial activity of SE484 spent broths against Bacillus subtilis. Indicated timepoints correspond to the time of growth where the spent media was collected, and a higher MIC value corresponds to more potent antimicrobial activity.

FIGS. 9A-9C depicts graphs showing the growth and active LEKTI production by SE351 formulated in oleyl alcohol:cetyl alcohol 1:1 (w/w) supplemented with 1% colloidal oatmeal (w/w) and 2% D-Alanine (w/w) and applied on ex vivo pig skin at (FIG. 9A) 10⁶, (FIG. 9B) 10⁷ and (FIG. 9C) 10⁸ CFU/cm². Indicated times represent the skin incubation period at 30° C. Dotted lines represented the average CFU/cm² (upper) and LEKTI activity (lower) found in placebo samples.

### DETAILED DESCRIPTION OF THE INVENTION

The present disclosure relates to a microbial product formulated in a non-aqueous composition designed to stabilize the bacterial product and increase protein expression upon administration. According to some embodiments, the dry aspect of the composition acts to stabilize the composition for storage. According to some embodiments, the composition increases protein expression after administration of the microbial product to a subject in of treatment of a disease or disorder.

On aspect of the present disclosure provides a composition, which increases protein expression of recombinant proteins, of Staphylococcus epidermidis, that is genetically altered to express recombinant proteins to treat or ameliorate a disease or disorder, e.g., a skin disease or disorder, inflammatory disease, and cancer.

As used herein the term “skin disease” and grammatical variations thereof means a skin state or condition that is generally undesirable or deleterious compared to the normal or baseline condition of human skin. Examples of abnormal skin conditions include, without limitation, Netherton Syndrome, psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism.

As used herein, the term “genetically modified” and grammatical variations thereof are used to describe a microbial organism (e.g., bacteria) that has been genetically modified or engineered by the introduction of DNA prepared outside the microbe. For example, the introduction of plasmid DNA containing new genes into bacteria will allow the bacteria to express those genes. Alternatively, the DNA containing new genes can be introduced to the bacteria and then integrated into the bacteria's genome, where the bacteria will express those genes.

As used herein, the term “microorganism” or “recombinant microorganism” refers to a microorganism, e.g., bacteria cell, that has been genetically modifies form its native state. Thus, a “recombinant bacterial cell” or “recombinant bacteria” refers to a bacterial cell or a bacteria that have been genetically modified form their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell. Recombinant bacterial cells may comprise exogenous nucleotide sequences stably incorporated into their chromosome.

As used herein, the terms “treat,” “treating,” “treatment” and grammatical variations thereof mean providing to a subject a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

In the present invention, the subject may be a mammal. As used herein, a “mammal” and grammatical variations thereof means any category of mammal. In the present invention, mammals include, for example, humans, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Preferably, the mammal is a human.

As used herein, the term “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a composition of the present invention may be administered as two, three, four, five, six or more sub-doses, administered separately at appropriate intervals throughout the day.

As used herein, the term “about” generally refers to a range that may be 1.5% greater or 1.5% less than the stated numerical value within the context of the particular usage. For example, “about 5” would include the range from 3.5 to 6.5.

According to some embodiments, the composition comprises a viable microorganism and a mixture of one or more disaccharides, one or more oligosaccharides, and one or more antioxidants. According to some embodiments, the composition further comprises one or more fatty alcohols. According to some embodiments, the composition further comprises colloidal oatmeal and/or alkaline oatmeal extract.

According to some embodiments, the bacteria suitable for use in the present invention include, but are not limited to is Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof. According to some embodiments, microbial compositions comprise one or more of Staphylococcus warneri, Streptococcus pyogenes, Streptococcus mitis, Propionibacterium acnes, Corynebacterium spp., Acinetobacter johnsonii, Pseudomonas aeruginosa. According to some embodiments, other related or similar species found on the skin are used. Certain embodiments involve the use of bacterium Staphylococcus epidermidis.

According to some embodiments, the composition comprises a mixture of one or more disaccharides selected from a group consisting of lactose, trehalose, sucrose, maltose, and cellobiose. According to some embodiments, the one or more oligosaccharides is selected from a group consisting of fructo-oligosaccharides (FOS), galactooligosaccharides (GOS), mannan oligosaccharides (MOS), isomalto-oligosaccharides, xylo-oligosaccharides, galacto-oligosaccharides, and raffinose. According to some embodiments, the composition comprises one or more antioxidants selected from the group consisting of ascorbic acid, vitamin A, vitamin E, a-carotene, lycopene, lutein, zeaxanthin, and water soluble derivatives of lipophilic antioxidants, e.g., alpha-tocopherol phosphate, alpha-tocopherol polyethylene glycol ester. According to one embodiment, the disaccharide is lactose, the oligosaccharide is fructo-oligosaccharides, and the antioxidant is ascorbic acid.

According to some embodiments, the microorganisms are mixed with the disaccharides, oligosaccharides, and antioxidants, and the composition further comprises one or more fatty alcohols selected from the group consisting of saturated fatty alcohol and unsaturated fatty alcohol. According to some embodiments, the saturated fatty alcohol is cetyl alcohol and the unsaturated fatty alcohol is oleyl alcohol. According to some embodiments, the cetyl alcohol and oleyl alcohol are at a 1:1 ratio.

According to some embodiments, the one or more disaccharides is about 20% (w/w), about 25% (w/w), about 30% (w/w), about 35% (w/w), about 40% (w/w), about 45% (w/w), about 50% (w/w), about 55% (w/w), or about 60% (w/w). According some embodiments, the one or more disaccharides is between about 20% (w/w) and about 30% (w/w), between about 25% (w/w) and about 35% (w/w), between about 30% (w/w) and about 40% (w/w), between about 35% (w/w) and about 45% (w/w), between about 40% (w/w) and about 50% (w/w), between about 45% (w/w) and about 55% (w/w), or between about 50% (w/w) and about 60% (w/w). According to some embodiments, the one or more disaccharides is at least 20% (w/w), at least 25% (w/w), at least 30% (w/w), at least 35% (w/w), at least 40% (w/w), at least 45% (w/w), at least 50% (w/w), at least 55% (w/w), or at least 60% (w/w). According to some embodiments, the one or more disaccharides is no more than 20% (w/w), no more than 25% (w/w), no more than 30% (w/w), no more than 35% (w/w), no more than 40% (w/w), no more than 45% (w/w), no more than 50% (w/w), no more than 55% (w/w), or no more than 60% (w/w).

According to some embodiments, the one or more oligosaccharides is about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), or about 20% (w/w). According some embodiments, the one or more oligosaccharides is between about 5% (w/w) and about 20% (w/w), between about 5% (w/w) and about 15% (w/w), between about 5% (w/w) and about 10% (w/w), between about 10% (w/w) and about 20% (w/w), between about 10% (w/w) and about 15% (w/w), or between about 15% (w/w) and about 20% (w/w). According to some embodiments, the one or more oligosaccharides is at least 5% (w/w), at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), or at least 20% (w/w). According to some embodiments, the one or more food source oligosaccharides is no more than 5% (w/w), no more than 6% (w/w), no more than 7% (w/w), no more than 8% (w/w), no more than 9% (w/w), no more than 10% (w/w), no more than 11% (w/w), no more than 12% (w/w), no more than 13% (w/w), no more than 14% (w/w), no more than 15% (w/w), no more than 16% (w/w), no more than 17% (w/w), no more than 18% (w/w), no more than 19% (w/w), or no more than 20% (w/w).

According to some embodiments, the one or more antioxidants is about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1.0% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), or about 2.0% (w/w). According some embodiments, the one or more antioxidants is between about 0.1% (w/w) and about 2.0% (w/w), between about 0.1% (w/w) and about 1.5% (w/w), between about 0.1% (w/w) and about 1.0% (w/w), between about 0.1% (w/w) and about 0.5% (w/w), between about 0.5% (w/w) and about 2.0% (w/w), between about 0.5% (w/w) and about 1.5% (w/w), between about 0.5% (w/w) and about 1.0% (w/w), between about 1.0% (w/w) and about 2.0%, or between about 1.0% (w/w) and about 1.5% (w/w). According to some embodiments, the one or more antioxidants is at least 0.1% (w/w), at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1.0% (w/w), at least 1.1% (w/w), at least 1.2% (w/w), at least 1.3% (w/w), at least 1.4% (w/w), at least 1.5% (w/w), at least 1.6% (w/w), at least 1.7% (w/w), at least 1.8% (w/w), at least 1.9% (w/w), or at least 2.0% (w/w). According to some embodiments, the one or more antioxidants is no more than 0.1% (w/w), no more than 0.2% (w/w), no more than 0.3% (w/w), no more than 0.4% (w/w), no more than 0.5% (w/w), no more than 0.6% (w/w), no more than 0.7% (w/w), no more than 0.8% (w/w), no more than 0.9% (w/w), no more than 1.0% (w/w), no more than 1.1% (w/w), no more than 1.2% (w/w), no more than 1.3% (w/w), no more than 1.4% (w/w), no more than 1.5% (w/w), no more than 1.6% (w/w), no more than 1.7% (w/w), no more than 1.8% (w/w), no more than 1.9% (w/w), or no more than 2.0% (w/w).

According to some embodiments, the one or more microorganisms is about 20% (w/w), about 25% (w/w), about 30% (w/w), about 35% (w/w), about 40% (w/w), or about 45% (w/w). According some embodiments, the one or more microorganisms is between about 20% (w/w) and about 45% (w/w), between about 20% (w/w) and about 40% (w/w), between about 20% (w/w) and about 35% (w/w), between about 20% (w/w) and about 30% (w/w), between about 20% (w/w) and about 25% (w/w), between about 25% (w/w) and about 45% (w/w), between about 25% (w/w) and about 40% (w/w), between about 25% (w/w) and about 35% (w/w), between about 25% (w/w) and about 30% (w/w), between about 30% (w/w) and about 45% (w/w), between about 30% (w/w) and about 40% (w/w), between about 30% (w/w) and about 35% (w/w), between about 35% (w/w) and about 45% (w/w), between about 35% (w/w) and about 40% (w/w), or between about 40% (w/w) and about 45% (w/w). According to some embodiments, the one or more microorganisms is at least 20% (w/w), at least 25% (w/w), at least 30% (w/w), at least 35% (w/w), at least 40% (w/w), or at least 45% (w/w). According to some embodiments, the one or more microorganisms is no more than 20% (w/w), no more than 25% (w/w), no more than 30% (w/w), no more than 35% (w/w), or no more than 40% (w/w), no more than 45% (w/w).

According to some embodiments, the microorganisms of the composition has a viability of at least 2.5×10¹⁰ CFU/g, at least 5×10¹⁰ CFU/g, at least 1×10¹¹ CFU/g, at least 2.5×10¹¹ CFU/g, at least 5×10¹¹ CFU/g, at least 1×10¹² CFU/g, at least 2.5×10¹² CFU/g, or at least 5×10¹² CFU/g. According to some embodiments, the microorganisms of the composition has a viability of about 2.5×10¹⁰ CFU/g, about 5×10¹⁰ CFU/g, about 1×10¹¹ CFU/g, about 2.5×10¹¹ CFU/g, about 5×10¹¹ CFU/g, about 1×10¹² CFU/g, about 2.5×10¹² CFU/g, or about 5×10¹² CFU/g. According to some embodiments, the microorganisms of the composition has a viability of between about 2.5×10¹⁰ CFU/g and about 2.5×10¹² CFU/g, between about 2.5×10¹⁰ CFU/g and about 1×10¹² CFU/g, between about 2.5×10¹⁰ CFU/g and about 5×10¹¹ CFU/g, between about 2.5×10¹⁰ CFU/g and about 2.5×10¹¹ CFU/g, between about 2.5×10¹⁰ CFU/g and about 1×10¹¹ CFU/g, between 2.5×10¹⁰ CFU/g and about 5×10¹⁰ CFU/g, between about 5×10¹⁰ CFU/g and about 2.5×10¹² CFU/g, between about 5×10¹⁰ CFU/g and about 1×10¹² CFU/g, between about 5×10¹⁰ CFU/g and about 5×10¹¹ CFU/g, between about 5×10¹⁰ CFU/g and about 2.5×10¹¹ CFU/g, between about 5×10¹⁰ CFU/g and about 1×10¹¹ CFU/g, between about 1×10¹¹ CFU/g and about 2.5×10¹² CFU/g, between about 1×10¹¹ CFU/g and about 1×10¹² CFU/g, between about 1×10¹¹ CFU/g and about 5×10¹¹ CFU/g, between about 1×10¹¹ CFU/g and about 2.5×10¹¹ CFU/g, between about 2.5×10¹¹ CFU/g and about 2.5×10¹² CFU/g, between about 2.5×10¹¹ CFU/g and about 1×10¹² CFU/g, between about 2.5×10¹¹ CFU/g and about 5×10¹¹ CFU/g, between about 5×10¹¹ CFU/g and about 2.5×10¹² CFU/g, between about 5×10¹¹ and about 1×10¹² CFU/g, between about 1×10¹² CFU/g and about 2.5×10¹² CFU/g, between about 2.5×10¹⁰ CFU/g and about 5×10¹² CFU/g, between about 5×10¹⁰ CFU/g and about 5×10¹² CFU/g, between about 1×10¹¹ CFU/g and about 5×10¹² CFU/g, between about 2.5×10¹¹ CFU/g and about 5×10¹² CFU/g, between about 5×10¹¹ CFU/g and about 5×10¹² CFU/g, or between 1×10¹² CFU/g and about 5×10¹² CFU/g.

According to some embodiments, the microorganism, disaccharides, oligosaccharides, and antioxidant mixture is at a ratio of about 10:1 with the mixture of fatty alcohols. According to some embodiments, the ratio between the microorganism, disaccharides, oligosaccharides, and antioxidant mixture and the fatty alcohol mixture is about 8:1, about 9:1, about 10:1, or about 11:1.

According to some embodiments, the composition further comprises colloidal oatmeal and/or alkaline oatmeal extract. According to some embodiments, the colloidal oatmeal and/or alkaline oatmeal extract are mixed with the fatty alcohols. According to some embodiments, the colloidal oatmeal is at about 0.1% (wt/wt), about 0.5% (wt/wt), about 1.0% (wt/wt), about 1.5% (wt/wt), or about 2.0% (wt/wt). According to some embodiments, the colloidal oatmeal is between about 0.1% (wt/wt) and about 2.0% (wt/wt), between about 0.1% (wt/wt) and about 1.5% (wt/wt), between about 0.1% (wt/wt) and about 1.0% (wt/wt), between about 0.1% (wt/wt) and about 0.5% (wt/wt), between 0.5% and about 2.0% (wt/wt), between about 0.5% (wt/wt) and about 1.5% (wt/wt), between 0.5% (wt/wt) and about 1.0% (wt/wt), between about 1.0% and about 2.0% (wt/wt), about 1.0% (wt/wt) and about 1.5% (wt/wt), or between about 1.5% (wt/wt) and about (2.0% (wt/wt). According to some embodiments, the colloidal oatmeal is at least 0.1% (wt/wt), at least 0.5% (wt/wt), at least 1.0% (wt/wt), at least 1.5% (wt/wt), or at least 2.0% (wt/wt). According to some embodiments, the colloidal oatmeal is no more than 0.1% (wt/wt), no more than 0.5% (wt/wt), no more than 1.0% (wt/wt), no more than 1.5% (wt/wt), or no more than 2.0% (wt/wt).

According to some embodiments, the alkaline oatmeal extract is about 0.1% (wt/wt), about 0.5% (wt/wt), about 1.0% (wt/wt), about 1.5% (wt/wt), or about 2.0% (wt/wt). According to some embodiments, the alkaline oatmeal extract is between about 0.1% (wt/wt) and about 2.0% (wt/wt), between about 0.1% (wt/wt) and about 1.5% (wt/wt), between about 0.1% (wt/wt) and about 1.0% (wt/wt), between about 0.1% (wt/wt) and about 0.5% (wt/wt), between 0.5% and about 2.0% (wt/wt), between about 0.5% (wt/wt) and about 1.5% (wt/wt), between 0.5% (wt/wt) and about 1.0% (wt/wt), between about 1.0% and about 2.0% (wt/wt), about 1.0% (wt/wt) and about 1.5% (wt/wt), or between about 1.5% (wt/wt) and about (2.0% (wt/wt). According to some embodiments, the alkaline oatmeal extract is at least 0.1% (wt/wt), at least 0.5% (wt/wt), at least 1.0% (wt/wt), at least 1.5% (wt/wt), or at least 2.0% (wt/wt). According to some embodiments, the alkaline oatmeal extract is no more than 0.1% (wt/wt), no more than 0.5% (wt/wt), no more than 1.0% (wt/wt), no more than 1.5% (wt/wt), or no more than 2.0% (wt/wt).

According to some embodiments, the composition is prepared by combining the disaccharides, oligosaccharides, antioxidant, and the viable microorganisms in to a first mixture and lyophilizing that mixture. According to some embodiments, the lyophilized first mixture comprising disaccharides, oligosaccharides, antioxidant, and the viable microorganisms is combined with the fatty alcohols, e.g., oleyl alcohol and cetyl alcohol. According to some embodiments, the fatty alcohols further comprise colloidal oatmeal and/or alkaline oatmeal extract.

According to some embodiments, the composition provides for increased protein expression when compared to a composition without fatty alcohols and/or colloidal oatmeal and/or alkaline oatmeal extract. According to some embodiments, the fold increase of protein expression when compared to a composition without fatty alcohols and/or colloidal oatmeal and/or alkaline oatmeal extract is about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, or about 15-fold.

According to some embodiments, the composition provides for increased protein activity of expressed protein when compared to a composition without fatty alcohols and/or colloidal oatmeal and/or alkaline oatmeal extract. According to some embodiments, the fold increase of protein activity of expressed protein when compared to a composition without fatty alcohols and/or colloidal oatmeal and/or alkaline oatmeal extract is about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, or about 15-fold.

According to some embodiments the composition for use according to the present invention can comprise any pharmaceutically effective amount of the recombinant bacteria to produce a therapeutically effective amount of the desired polypeptide or therapeutically effective domain(s) thereof, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about. 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of recombinant bacteria, the upper limit of which is about 90.0% by weight of recombinant, bacteria.

According to some embodiments, the composition for use according to the present invention can comprise, for example, at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, or more by weight of recombinant bacteria.

### Microorganisms and Microbial Compositions

The genetically altered protein-producing microorganism are able to treat a disease or disorder by expressing and, optionally, secreting a therapeutic protein that treats the underlying cause of the disease or disorder or its symptoms. According to some embodiments, the therapeutic proteins is a LEKTI protein domain, filaggrin, interferons, or interleukins.

According to some embodiments, the therapeutic protein comprises one or more LEKTI domains that are effective to inhibit serine proteases within or on the skin of a mammal According to some embodiments, the recombinant LEKTI domains compensate for the defective endogenous LEKTI protein naturally produced in the mammal According to some embodiments, the genetically altered bacteria are able to self-replicate while retaining the ability to produce the recombinant protein, thereby providing a continuous supply of therapeutic agent.

According to some embodiments, the disclosure provides a composition for a microbial product for the treatment of a disease or disorder that comprising a microbe genetically modified to express and provide one or more LEKTI protein domains to a target site in a mammal, wherein the LEKTI protein domains are effective to penetrate one or more layers of the mammal's skin and effective to inhibit serine protease activity of at least one serine protease in or on the mammal's skin.

As used herein, the term “auxotrophic” or “auxotrophy” refers to inability of an organism to synthesize a particular compound required for its growth. An auxotroph is an organism that displays this characteristic.

As used herein, the term “alrA” and “alr” refer to the D-alanine racemase gene, including normal alleles of the alrA gene. In some embodiments, the alr gene from S. epidermidis (UniProtKB—Q8CNK7 (ALR_STAES) encodes a D-alanine racemase protein (EC 5.1.1.1). In some embodiments, the locus identifiers SE1674 (alrl) and SE1079 (alr2) refer to specific S. epidermidis D-alanine racemase genes.

As used herein, the term “dat” refers to the D-alanine aminotransferase gene, including normal alleles of the dat gene. In some embodiments, the dat gene from S. epidermidis (UniProtKB—Q8CS41 (DAAA_STAES)) encodes a D-alanine aminotransferase protein (EC:2.6.1.21). In some embodiments, the locus identifier SE1423 (dat) refers to a specific S. epidermidis D-alanine aminotransferase gene. As used herein, the term “murI” refers to the glutamate racemase gene, including normal alleles of the murI gene. In some embodiments, the murI gene from S. epidermidis (UniProtKB—Q8CPL0 (MURI_STAES)) encodes a glutamate racemase protein (EC:5.1.1.3). In some embodiments, the locus identifier SE0843 (murI) refers to a specific S. epidermidis glutamate racemase gene.

D-alanine auxotrophs of S. aureus have been produced for the purpose of producing vaccines MRSA. (Moscoso M, et al. 27th ECCMID 22-25 Apr. 2017, The Congress of ESCMID (P0473); Moscoso et al., Virulence (2018) Vol. 9(1): 604-620, the contents of each being incorporated by reference in its entirety herein). In this case, it was found necessary to knockout only the alanine racemase alrl, and also a dat gene.

According to some embodiments, the disclosure provides a recombinant Staphylococcus epidermidis strain containing a DNA cassette in its chromosome which expresses a secreted form of the domain 6 of LEKTI protein (hLEKTI-d6) of the human SPINKS gene. The host strain harboring this expression cassette in its chromosome is S. epidermidis strain SE_{ΔΔΔ}, a strain containing deletions of three genes involved in the biosynthesis of the essential amino acid, D-alanine [two alanine racemase genes (alr1 and alr2) and the D-alanine aminotransferase gene (dat)], making the strain auxotrophic to this amino acid.

According to some embodiments, the chromosomal hLEKTI-d6 construct strain possesses several differentiating properties as compared to another strain, S. epidermidis 27a, which is S. epidermidis SE_{ΔΔΔ} expressing secreted hLEKTI-d6 protein from a plasmid construct that also contains an alrA gene complementing D-alanine auxotrophy. These properties include: 1) retention of the D-alanine auxotrophy of SE_{ΔΔΔ}, providing a measure of growth control using D-alanine supplementation; 2) an unexpected enhanced production of secreted hLEKTI-d6 protein, presumably influenced by the genetic environment at the chromosomal site of integration, enhanced levels of secreted hLEKTI-d6 protein may be due to a synergy of promoter combinations driving hLEKTI-d6 gene expression (1; 2), activation of transcription triggered by quorum sensing in dense cultures (3; 4), and/or unique DNA or RNA structure associated with regulation of the delta-toxin gene, hld (4); and 3) potential enhanced stability of the expression cassette, and reduced potential for lateral transmission to commensal flora, due to the chromosomally integrated nature of the expression cassette vs. being carried on an extrachromosomal genetic element (plasmid).

According to some embodiments, the disclosure provides microbial compositions comprising one or more of a wide range of bacteria suitable for use on a mammal's skin. Examples include, but are not limited to, non-pathogenic and commensal bacteria. Bacteria suitable for use in the present invention include, but are not limited to, Bifidobacterium, Brevibacterium, Propionibacterium, Lactococcus, Streptococcus, Staphylococcus (e.g., S. epidermidis and/or S. hominis), Lactobacillus (e.g., L. acidophilus), Pediococcus, Leuconostoc, or Oenococcus. According to some embodiments, microbial compositions comprise one or more of Staphylococcus warneri, Streptococcus pyogenes, Streptococcus mitis, Propionibacterium acnes, Corynebacterium spp., Acinetobacter johnsonii, Pseudomonas aeruginosa. According to some embodiments, other related or similar species found on the skin are used.

Certain embodiments involve the use of bacterium Staphylococcus epidermidis. According to some embodiments, the strain of S. epidermidis to be used is incapable of producing biofilms. An example of this is S. epidermidis strain ATCC 12228 or NRRL B-4268.

According to some embodiments, the recombinant microbe is adapted to live indefinitely or for a controlled duration on the surface of the mammal's skin to provide a continuous supply of LEKTI protein domains. In some embodiments, the recombinant microbe lives alongside commensal microorganisms naturally occurring on the mammal's skin. In some embodiments, the recombinant microbe lives to the exclusion of commensal microorganisms that naturally occur on the mammal's skin. According to some embodiments, the recombinant microbe is adapted to multiply on the skin of the mammal. In other embodiments, the recombinant microbe is no longer alive, but contains effective amounts of a therapeutic polypeptide, e.g., LEKTI or therapeutically effective domain(s) thereof. Such cells may be intact or not depending upon the particulars of delivering the therapeutic peptide (or domain(s) thereof) to the target site.

As used herein, the term “recombinant” and grammatical variations thereof means relating to or denoting an organism, protein, or genetic material formed by or using recombined DNA comprising DNA pieces from different sources or from different parts of the same source. For example, the term “recombinant DNA” means a DNA molecule formed through recombination methods to splice fragments of DNA from a different source or from different parts of the same source. In some embodiments, two or more different sources of DNA are cleaved using restriction enzymes and joined together using ligases. As another example, the term “recombinant protein” or “recombinant domains” and grammatical variations thereof means a protein molecule formed through recombination methods originating from spliced fragments of DNA from a different source or from different parts of the same source. As another example, the term “recombinant microbe” or “recombinant bacteria” and grammatical variations thereof mean a microbe/bacteria that comprises one or more recombinant DNA/protein molecules.

LEKTI gene: According to some embodiments, the recombinant microbe is engineered to express a mammalian gene encoding LEKTI protein. The LEKTI gene can be obtained from any mammal, such as mouse, rat, rabbit, goat, sheep, horse, cow, dog, primate, or human gene sequences. According to some embodiments, the LEKTI gene sequence is a human gene sequence. According to some embodiments, the recombinant microbe is engineered to comprise a fragment of the LEKTI gene.

According to some embodiments, the recombinant microbe comprises a sequence as disclosed herein that has at least about 75% identity, or 80% identity, or 85% identity, or 90% identity, or 95% identity to any one or more of the SEQ ID NOS listed herein. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

According to some embodiments, the recombinant protein expressed by the engineered microbe comprises one or more protease inhibitory domains of the LEKTI protein. Some non-limiting examples include one or more of domains D1, D2, D3, D4, D5, D6, D7, D8, D9, D10, D11, D12, D13, D14, and D15. According to some embodiments, the recombinant protein expressed by the engineered microbe comprises LEKTI inhibitory domain 6 or domains D8 to D11.

According to some embodiments, the LEKTI protein domains are effective to ameliorate the symptoms of Netherton Syndrome. As used herein, the terms “ameliorate”, “ameliorating” and grammatical variations thereof mean to decrease the severity of the symptoms of a disease in a subject. In some embodiments, the LEKTI protein domains act as a competitive or non-competitive inhibitor of one or more proteases present on or in the skin of a mammal. In some embodiments, the LEKTI protein domain acts as a serine protease inhibitor. As used herein, the terms “protease” and “proteinase” are used interchangeably, with both terms referring to an enzyme that performs proteolysis.

According to some embodiments, the microbe is genetically modified by transfection/transformation with a recombinant DNA plasmid encoding the LEKTI protein domains. Other conventional or to-be-discovered methods for introducing DNA into a microbe may also be used in the present invention. According to some embodiments, the recombinant DNA plasmid comprises sequences encoding the LEKTI protein domain and one or more secretory peptides and/or cell penetration peptides. According to some embodiments, the LEKTI domains are operably linked to one or more recombinant protein domains that are effective to enhance secretion from the microbe and/or penetration of the mammal's skin.

The present disclosure also relates to allelic variants of LEKTI, or portions thereof (one or more of domains D1, D2, D3, D4, D5, D6, D7, D8, D9, D10, D11, D12, D13, D14, and D15), as well as synthetic or mutated genes of SPINK (e.g., SPINKS) that have been modified to change, for example, the expression or activity of the recombinant protein. It is also noted that degeneracy of the nucleic acid code can be considered variations in the nucleotide sequences that encode the same amino acid residues. Accordingly, the disclosure includes nucleic acid residues that are able to hybridize under moderately stringent conditions. One skilled in the art can determine effective combinations of salt and temperature to constitute a moderately stringent hybridization condition. It is also envisioned that orthologs of LEKTI are present in other species, for example, dog, sheep, rat, hamster, chicken and pig. Therefore in another embodiment of the present invention relates to SPINK (e.g., SPINKS) nucleic acids that encode polypeptides having at least about 70% to 80% identity, preferably 90% to 95% identity, more preferably 98% to 99% identity to LEKTI set forth in SEQ ID NO: 103 or portions thereof (one or more of domains D1, D2, D3, D4, D5, D6, D7, D8, D9, D10, D11, D12, D13, D14, and D15).

According to some embodiments, the LEKTI domains are selected from the non-limiting examples below, Tables 1 and 2.

TABLE 1
LEKTI Amino Acid Sequences

| | | SEQ |
|---|---|---|
| | | ID |
| LEKTI Sequence | Amino acid sequence | NO: |
| LEKTI amino acid | MKIATVSVLLPLALCLIQDAASKNEDQEMCHEFQAFMKNGKLFCPQDKKF | 103 |
| sequence | FQSLDGIMFINKCATCKMILEKEAKSQKRARHLARAPKATAPTELNCDDE | |
| Residues 1-1064 | KKGERDGDFICPDYYEAVCGTDGKTYDNRCALCAENAKTGSQIGVKSEGE | |
| | CKSSNPEQDVCSAFRPFVRDGRLGCTRENDPVLGPDGKTHGNKCAMCAEL | |
| | FLKEAENAKREGETRIRRNAEKDFCKEYEKQVRNGRLFCTRESDPVRGPD | |
| | GRMHGNKCALCAEIFKQRFSEENSKTDQNLGKAEEKTKVKREIVKLCSQY | |
| | QNQAKNGILFCTRENDPIRGPDGKMHGNLCSMCQAYFQAENEEKKKAEAR | |
| | ARNKRESGKATSYAELCSEYRKLVRNGKLACTRENDPIQGPDGKVHGNTC | |
| | SMCEVFFQAEEEEKKKKEGKSRNKRQSKSTASFEELCSEYRKSRKNGRLF | |
| | CTRENDPIQGPDGKMHGNTCSMCEAFFQQEERARAKAKREAAKEICSEFR | |
| | DQVRNGTLICTREHNPVRGPDGKMHGNKCAMCASVFKLEEEEKKNDKEEK | |
| | GKVEAEKVKREAVQELCSEYRHYVRNGRLPCTRENDPIEGLDGKIHGNTC | |
| | SMCEAFFQQEAKEKERAEPRAKVKREAEKETCDEFRRLLQNGKLFCTREN | |
| | DPVRGPDGKTHGNKCAMCKAVFQKENEERKRKEEEDQRNAAGHGSSGGGG | |
| | GNTQDECAEYREQMKNGRLSCTRESDPVRDADGKSYNNQCTMCKAKLERE | |
| | AERKNEYSRSRSNGTGSESGKDTCDEFRSQMKNGKLICTRESDPVRGPDG | |
| | KTHGNKCTMCKEKLEREAAEKKKKEDEDRSNTGERSNTGERSNDKEDLCR | |
| | EFRSMQRNGKLICTRENNPVRGPYGKMHINKCAMCQSIFDREANERKKKD | |
| | EEKSSSKPSNNAKDECSEFRNYIRNNELICPRENDPVHGADGKFYTNKCY | |
| | MCRAVELTEALERAKLQEKPSHVRASQEEDSPDSFSSLDSEMCKDYRVLP | |
| | RIGYLCPKDLKPVCGDDGQTYNNPCMLCHENLIRQTNTHIRSTGKCEESS | |
| | TPGTTAASMPPSDE | |
| LEKTI Domain 1 | KNEDQEMCHEFQAFMKNGKLFCPQDKKFFQSLDGIMFINKCATCKMILEK | 104 |
| (residues 23-77) | EAKSQ | |
| LEKTI Domain 2 | APTELNCDDFKKGERDGDFICPDYYEAVCGTDGKTYDNRCALCAENAKTG | 105 |
| (residues 91-153) | SQIGVKSEGECKS | |
| LEKTI Domain 3 | NPEQDVCSAFRPFVRDGRLGCTRENDPVLGPDGKTHGNKCAMCAELFLKE | 106 |
| (residues 155-216 | AENAKREGETRI | |
| LEKTI Domain 4 | NAEKDFCKEYEKQVRNGRLFCTRESDPVRGPDGRMHGNKCALCAEIFKQR | 107 |
| (residues 219- | FSEENSKTDQNLGKAEE | |
| 285) | | |
| LEKTI_Domain 5 | REIVKLCSQYQNQAKNGILFCTRENDPIRGPDGKMHGNLCSMCQAYFQAE | 108 |
| (residues 291- | NEEKKKAEARAR | |
| 352) | | |
| LEKTI_Domain 6 | ESGKATSYAELCSEYRKLVRNGKLACTRENDPIQGPDGKVHGNTCSMCEV | 109 |
| (residues 356- | FFQAEEEEKKKKEGKSRN | |
| 423) | | |
| LEKTI Domain 7 | ASFEELCSEYRKSRKNGRLFCTRENDPIQGPDGKMHGNTCSMCEAFFQQE | 110 |
| (residues 431- | ERARAKAKR | |
| 489) | | |
| LEKTI Domain 8 | EAAKEICSEFRDQVRNGTLICTREHNPVRGPDGKMHGNKCAMCASVFKLE | 111 |
| (residues 490- | EEEKKNDKEEKG | |
| 550) | | |
| LEKTI Domain 9 | EAVQELCSEYRHYVRNGRLPCTRENDPIEGLDGKIHGNTCSMCEAFFQQE | 112 |
| (residues 561- | AKEKERAEPRAK | |
| 622) | | |
| LEKTI Domain | EAEKETCDEFRRLLQNGKLFCTRENDPVRGPDGKTHGNKCAMCKAVFQKE | 113 |
| 10 (residues 626- | NEERKRKEEEDQR | |
| 688) | | |
| LEKTI Domain | GNTQDECAEYREQMKNGRLSCTRESDPVRDADGKSYNNQCTMCKAKLERE | 114 |
| 11 (residues 701- | AERKNEY | |
| 757) | | |
| LEKTI Domain | ESGKDTCDEFRSQMKNGKLICTRESDPVRGPDGKTHGNKCTMCKEKLERE | 115 |
| 12 (residues 768- | AAEKKKKEDEDRS | |
| 830) | | |
| LEKTI Domain | NDKEDLCREFRSMQRNGKLICTRENNPVRGPYGKMHINKCAMCQSIFDRE | 116 |
| 13 (residues 843- | ANERKKKDEEKSS | |
| 905) | | |
| LEKTI Domain | NNAKDECSEFRNYIRNNELICPRENDPVHGADGKFYTNKCYMCRAVELTE | 117 |
| 14 (residues 910- | ALERAKLQEKPS | |
| 970) | | |
| LEKTI Domain | SLDSEMCKDYRVLPRIGYLCPKDLKPVCGDDGQTYNNPCMLCHENLIRQT | 118 |
| 15 (residues 987- | NTHIRSTGKCEE | |
| 1048) | | |

TABLE 2
SPINK5 Nucleotide Sequence

LEKTI Full length Nucleic acid sequence
(SEQ ID NO: 119)
ATGAAGATAGCCACAGTGTCAGTGCTTCTGCCCTTGGCTCTTTGC
CTCATACAAGATGCTGCCAGTAAGAATGAAGATCAGGAAATGTGC
CATGAATTTCAGGCATTTATGAAAAATGGAAAACTGTTCTGTCCC
CAGGATAAGAAATTTTTTCAAAGTCTTGATGGAATAATGTTCATC
AATAAATGTGCCACGTGCAAAATGATACTGGAAAAAGAAGCAAAA
TCACAGAAGAGGGCCAGGCATTTAGCAAGAGCTCCCAAGGCTACT
GCCCCAACAGAGCTGAATTGTGATGATTTTAAAAAAGGAGAAAGA
GATGGGGATTTTATCTGTCCTGATTATTATGAAGCTGTTTGTGGC
ACAGATGGGAAAACATATGACAACAGATGTGCACTGTGTGCTGAG
AATGCGAAAACCGGGTCCCAAATTGGTGTAAAAAGTGAAGGGGAA
TGTAAGAGCAGTAATCCAGAGCAGGATGTATGCAGTGCTTTTCGG
CCCTTTGTTAGAGATGGAAGACTTGGATGCACAAGGGAAAATGAT
CCTGTTCTTGGTCCTGATGGGAAGACGCATGGCAATAAGTGTGCA
ATGTGTGCTGAGCTGTTTTTAAAAGAAGCTGAAAATGCCAAGCGA
GAGGGTGAAACTAGAATTCGACGAAATGCTGAAAAGGATTTTTGC
AAGGAATATGAAAAACAAGTGAGAAATGGAAGGCTTTTTTGTACA
CGGGAGAGTGATCCAGTCCGTGGCCCTGACGGCAGGATGCATGGC
AACAAATGTGCCCTGTGTGCTGAAATTTTCAAGCAGCGTTTTTCA
GAGGAAAACAGTAAAACAGATCAAAATTTGGGAAAAGCTGAAGAA
AAAACTAAAGTTAAAAGAGAAATTGTGAAACTCTGCAGTCAATAT
CAAAATCAGGCAAAGAATGGAATACTTTTCTGTACCAGAGAAAAT
GACCCTATTCGTGGTCCAGATGGGAAAATGCATGGCAACTTGTGT
TCCATGTGTCAAGCCTACTTCCAAGCAGAAAATGAAGAAAAGAAA
AAGGCTGAAGCACGAGCTAGAAACAAAAGAGAATCTGGAAAAGCA
ACCTCATATGCAGAGCTTTGCAGTGAATATCGAAAGCTTGTGAGG
AACGGAAAACTTGCTTGCACCAGAGAGAACGATCCTATCCAGGGC
CCAGATGGGAAAGTGCATGGCAACACCTGCTCCATGTGTGAGGTC
TTCTTCCAAGCAGAAGAAGAAGAAAAGAAAAAGAAGGAAGGTAAA
TCAAGAAACAAAAGACAATCTAAGAGTACAGCTTCCTTTGAGGAG
TTGTGTAGTGAATACCGCAAATCCAGGAAAAACGGACGGCTTTTT
TGCACCAGAGAGAATGACCCCATCCAGGGCCCAGATGGAAAAATG
CATGGCAACACCTGCTCCATGTGTGAGGCCTTCTTTCAACAAGAA
GAAAGAGCAAGAGCAAAGGCTAAAAGAGAAGCTGCAAAGGAAATC
TGCAGTGAATTTCGGGACCAAGTGAGGAATGGAACACTTATATGC
ACCAGGGAGCATAATCCTGTCCGTGGCCCAGATGGCAAAATGCAT
GGAAACAAGTGTGCCATGTGTGCCAGTGTGTTCAAACTTGAAGAA
GAAGAGAAGAAAAATGATAAAGAAGAAAAAGGGAAAGTCGAGGCT
GAAAAAGTTAAGAGAGAAGCAGTTCAGGAGCTGTGCAGTGAATAT
CGTCATTATGTGAGGAATGGACGACTCCCCTGTACCAGAGAGAAT
GATCCTATTGAGGGTCTAGATGGGAAAATCCACGGCAACACCTGC
TCCATGTGTGAAGCCTTCTTCCAGCAAGAAGCAAAAGAAAAAGAA
AGAGCTGAACCCAGAGCAAAAGTCAAAAGAGAAGCTGAAAAGGAG
ACATGCGATGAATTTCGGAGACTTTTGCAAAATGGAAAACTTTTC
TGCACAAGAGAAAATGATCCTGTGCGTGGCCCAGATGGCAAGACC
CATGGCAACAAGTGTGCCATGTGTAAGGCAGTCTTCCAGAAAGAA
AATGAGGAAAGAAAGAGGAAAGAAGAGGAAGATCAGAGAAATGCT
GCAGGACATGGTTCCAGTGGTGGTGGAGGAGGAAACACTCAGGAC
GAATGTGCTGAGTATCGGGAACAAATGAAAAATGGAAGACTCAGC
TGTACTCGGGAGAGTGATCCTGTACGTGATGCTGATGGCAAATCG
TACAACAATCAGTGTACCATGTGTAAAGCAAAATTGGAAAGAGAA
GCAGAGAGAAAAAATGAGTATTCTCGCTCCAGATCAAATGGGACT
GGATCAGAATCAGGGAAGGATACATGTGATGAGTTTAGAAGCCAA
ATGAAAAATGGAAAACTCATCTGCACTCGAGAAAGTGACCCTGTC
CGGGGTCCAGATGGCAAGACACATGGCAATAAGTGTACTATGTGT
AAGGAAAAACTGGAAAGGGAAGCAGCTGAAAAAAAAAAGAAAGAG
GATGAAGACAGGAGCAATACAGGAGAAAGGAGCAATACAGGAGAA
AGGAGCAATGACAAAGAGGATCTGTGTCGTGAATTTCGAAGCATG
CAGAGAAATGGAAAGCTTATCTGCACCAGAGAAAATAACCCTGTT
CGAGGCCCATATGGCAAGATGCACATCAATAAATGTGCTATGTGT
CAGAGCATCTTTGATCGAGAAGCTAATGAAAGAAAAAAGAAAGAT
GAAGAGAAATCAAGTAGCAAGCCCTCAAATAATGCAAAGGATGAG
TGCAGTGAATTTCGAAACTATATAAGGAACAATGAACTCATCTGC
CCTAGAGAGAATGACCCAGTGCACGGTGCTGATGGAAAGTTCTAT
ACAAACAAGTGCTACATGTGCAGAGCTGTCTTTCTAACAGAAGCT
TTGGAAAGGGCAAAGCTTCAAGAAAAGCCATCCCATGTTAGAGCT
TCTCAAGAGGAAGACAGCCCAGACTCTTTCAGTTCTCTGGATTCT
GAGATGTGCAAAGACTACCGAGTATTGCCCAGGATAGGTTATCTT
TGTCCAAAGGATTTAAAGCCTGTCTGTGGTGACGATGGCCAAACC
TACAACAATCCTTGCATGCTCTGTCATGAAAACCTGATACGCCAA
ACAAATACACACATCCGCAGTACAGGGAAGTGTGAGGAGAGCAGC
ACCCCAGGAACCACCGCAGCCAGCATGCCCCCGTCTGACGAA

According to some embodiments, the disease or disorder is selected from a skin disease, a disease or disorder associated with pain, cancer, and a viral infection. According to some embodiments, the skin disease is selected from the group consisting of pruritus, rosacea, psoriasis, atopic dermatitis, Ichthyosis vulgaris, and Netherton Syndrome. According to some embodiments, the disease or disorder associated with pain is selected from the group consisting of acute pain, chronic pain, nociceptive pain, neuropathic pain, traumatic pain, inflammatory pain, post-operative incision pain, pain associated with cancer, fracture pain, osteoporotic pain, bone cancer pain, and gout joint pain. According to some embodiments, the cancer is selected from the group consisting of malignant melanoma, colon cancer, breast cancer, lung cancer, ovarian cancer, gastric cancer, oral tongue squamous cell carcinoma, squamous cell cancer, prostate cancer, pancreatic cancer, liver cancer, kidney cancer, bladder cancer, cervical cancer, endometrial cancer, gallbladder cancer, brain cancer and oral cancer. According to some embodiments, the viral infection is selected from a group consisting of a respiratory infection, dermal infection, and a viral infection that causes cancer in a subject.

Antimicrobial agents: The term “antimicrobial agent”, “antimicrobial protein” or “antimicrobial polypeptide” as used herein can be used interchangeably and refers to any entity with antimicrobial activity, i.e., the ability to inhibit the growth and/or kill bacterium and/or fungus, e.g., Gram positive and Gram negative bacteria and fungus.

An antimicrobial agent is any agent which results in inhibition of growth or reduction of viability of a bacteria and/or fungus by at least about 30%, at least about 40%, at least about 50%, at least about 60%, at least about 70%, or more than 70%, or any integer between 30% and 70% or more, as compared to in the absence of the antimicrobial agent. Stated another way, an antimicrobial agent is any agent which reduces a population of bacterial and/or fungal cells, by at least about 30%, at least about 40%, at least about 50%, at least about 60%, at least about 70%, or more than 70%, or any integer between 30% and 70% as compared to in the absence of the antimicrobial agent. In one embodiment, an antimicrobial agent is an agent which specifically targets a bacteria cell. In another embodiment, an antimicrobial agent modifies (i.e. inhibits or activates or increases) a pathway which is specifically expressed in bacterial cells. In some embodiments, the antimicrobial agent is a polypeptide, i.e., a polypeptide expressed and secreted by the engineering microorganism.

Antimicrobial agents can include chitinase, a glucanase, or a peptidoglycan hydrolase.

As used herein, the term “chitinase” refers to an enzyme capable of catalyzes the hydrolysis of the β-1,4 linked N-acetylglucosamine polymers that form chitin chains, a major component of fungal cell walls. Chitinases are expressed in plants in response to pathogens.

As used herein, the term “glucanase” refers to an enzyme capable of catalyzing the degradation or depolymerization of complex carbohydrates. A glucanase in the composition may be capable of degrading one or more of cellooligosaccharide, lignocellulose, cellulose, hemicellulose, and pectin. Such enzymatic activity may be, but is not limited to, endoglucanase, exoglucanase, β-glucosidase, cellobiohydrolase, endo-1,4-β-xylanase, β-xylosidase, α-glucuronidase, α-L-arabinofuranosidase, acetylesterase, acetylxylanesterase, α-amylase, β-amylase, glucoamylase, pullulanase, β-glucanase, hemicellulase, arabinosidase, mannanase, pectin hydrolase, or pectate lyase activities. The glucanase of the composition may be capable of degrading one or more of beta-glucan, cellulose, cellobiose, pNP-D-glucopyranoside and xylan.

As used herein, the term “peptidoglycan hydrolase” refer to an enzyme that can degrade bacterial cell walls when exposed externally. The bacterial cell wall consists of glycan strands which are cross-linked by flexible peptide side chains, providing strength and rigidity to the bacterial cell wall. The peptidoglycan of both the Gram-positive and Gram-negative bacteria features repeating units of N-acetylglucosamine (NAG) and 3-(1-4)-N-acetylmuramic acid (NAM) cross-linked by peptide stem chains attached to NAM residues. The so-called peptidoglycan hydrolases (PGHs) are the enzymes responsible for cleaving the bonds within the peptidoglycan chains and side-chains branches.

The term “infection” or “microbial infection” which are used interchangeably herein refers to in its broadest sense, any infection caused by a microorganism and includes bacterial infections, fungal infections, yeast infections and protozomal infections.

Formulations: According to some embodiments, the topical formulation can be in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. According to some embodiments, the formulation can include a living cell culture composition and can comprise at least one engineered bacterial strain that produces a therapeutically effective recombinant polypeptide or therapeutically effective domain(s) thereof. This engineered living cell culture composition can deliver the polypeptide directly to the skin for treating or preventing abnormal skin conditions.

Topical formulations include those in which any other active ingredient(s) is (are) dissolved or dispersed in a dermatological vehicle known in the art (e.g. aqueous or nonaqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed can contain one or more components (for example, when the formulation is an aqueous gel, components in addition to water) selected from the following list: a solubilizing agent or solvent (e.g. a β-cyclodextrin, such as bydroxypropyl β-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A pharmaceutically acceptable carrier can also be incorporated in the formulation of the present invention and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation, “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

A film former, when it dries, forms a protective film over the site of application. The film inhibits removal of the active ingredient and keeps it in contact with the site being treated. An example of a film former that is suitable for use in this invention is Flexible Collodion, US P. As described in Remington: The Science and Practice of Pharmacy, 19th Ed. (Easton, PA: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the ease of applying a more fluid composition. It is generally necessary that the insoluble matter in a lotion be finely-divided.

Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred “organic macromolecules,” i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthalate, and methylcellulose; gums such as tragacanth and xanthan gum; sodium alginate; and gelatin, In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, PA: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxy-methylcellulose or the like as a base.

Enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter 6 of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present invention in addition to those identified above. These include, but are not limited to, antioxidants, astringents, perfumes, preservatives, emollients, pigments, dyes, humectants, propeliants, and sunscreen agents, as well as other classes of materials whose presence can be pharmaceutically or otherwise desirable. Typical examples of optional additives for inclusion in the formulations of the invention are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), α-tocopherol (Vitamin E), β-tocopherol, γ-tocopherol, δ-tocopherol, ε-tocopherol, ζι-tocopherol, Z^{Λ}-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), α-tocopherol (Vitamin E), β-tocopherol, γ-tocopherol, 6-tocopherol, ε-tocopherol, ζι-tocopherol, ζ₂-tocopherol, η-tocopherol, and retinol (Vitamin A), and/or pharmaceutically acceptable salts, esters, amides, or other derivatives thereof. A preferred tocopherol compound is a-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padimate O, phenylbenzirmdazole sulfonic acid, sulisobenzone, titanium dioxide, trolamine salicylate, zinc oxide, ensulizole, meradiraate, octinoxate, octisalate, and octocrylene. See Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from about 0.01% to about 20% of the total weight of the topical formulation.

The formulations of the invention can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds.

Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. In other embodiments, other agents can also be added, such as repressors and inducers, i.e., to inhibit (i.e. glycose) or induce (i.e. xylose) the production of the polypeptide of interest. Such additives can be employed provided they are compatible with and do not interfere with the function of the formulations.

The formulations can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the invention will be applied in the range of once or twice weekly up to once or twice daily.

The pharmaceutical compositions of the invention comprise one or more active ingredients, e.g. therapeutic agents, in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, salicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxy-methylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl-cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyimide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

The following examples are provided to further illustrate the methods of the present invention. These examples are illustrative only and are not intended to limit the scope of the invention in any way.

### EXAMPLES

The following examples are provided to further illustrate the methods of the present invention. These examples are illustrative only and are not intended to limit the scope of the invention in any way.

### Example 1

### Bacteria

Each strain LEKTI-D6-expressing strain is S. epidermidis SE_{ΔΔΔ} transformed with a different derivative of pUBTR119 expressing hLEKTI-D6 from a different set of promoters. The plasmids constructs were made as described below.

The original pUBTR119 plasmid comprises a Kan-R gene and triple promoter expression cassette including: 1) hpaII native promotor essential for plasmid replication, 2) phosphate-starvation inducible yxiE promoter, and 3) constitutive sarA promoter with the open reading frame (ORF) of SsaA1 secretion signal fused to the expressed protein downstream.

To make plasmid construct #27e, expressing hLEKT1-D6 under the control of an inducible xylA promoter, PxylA. A set of overlapping primers, designed for Gibson assembly protocol, SEQ ID NO: 1 and SEQ ID NO: 2 for vector, and SEQ ID NO: 3, SEQ ID NO: 4 and SEQ ID NO: 5 (Table 3) for inserts were used, designed and synthesized by IDT, Inc. (Gibson et al., Nature Methods 6:343-345 (2009)). The backbone of pUBTR (Km-, without kanamycin resistance gene) plasmid containing the xylR/PxylA promoter was used. yfhKSP-pro-LEKTI-D6-6xHis in a pRKK-Blue vector was used as a template to amplify the ORF of the recombinant protein. Q5® high-fidelity (HiFi) polymerase (NEB, Inc., Ipswich, MA) was used for all PCR reactions. PCR products were purified from 0.7% agarose gel by a freeze-thaw method and were used in NEBuilder® HiFi DNA assembly reactions. The entire HiFi reactions (20 μl) were incubated with 220 μl of Bacillus subtilis competent cells at 37° C. for 90 min, and plated on Luria-Bertani (LB) agar plates for overnight incubation at 37° C. Plasmid DNA were purified 4 ml overnight cultures in LB that were inoculated from resulted B. subtilis colonies. Constructs were sequence-verified through the ORF of xylR repressor to stop-codon of the LEKTI-D6 ORF. Stocks were made in 20% glycerol and stored at −80° C. from the sequence-verified cultures. Plasmid DNAs purified from B. subtilis were transformed into competent cells of S. epidermidis SE_{ΔΔΔ}. Four SE_{ΔΔΔ} transformants were fully sequence-verified and deposited at −80° C.

TABLE 3
Primers used for designing
plasmid constructs

| SEQ | | | |
|---|---|---|---|
| ID | | | |
| NO: | Name | bp | Sequence |
| 1 | RBSsod-3′-R | 26 | CAT AAA TAA |
| | | | TCA TCC TCC |
| | | | TAA GGT AC |
| 2 | 3stops-GA-F | 33 | AAT GAA TAA |
| | | | CGG ATC ATC |
| | | | TCC TGC AGG |
| | | | CAG CAC |
| 3 | LEKT1d6-3′- | 42 | GGA GAT GAT |
| | 3stops-R | | CCG TTA TTC |
| | | | ATT AAT TTC |
| | | | GAC |
| | | | TTT TGC CCT |
| | | | CCT |
| 4 | GA-RBSsod- | 47 | TAC CTT AGG |
| | yfhKSP-F | | AGG ATG ATT |
| | | | ATT TAT GAA |
| | | | AAA |
| | | | GAA ACA AGT |
| | | | AAT GCT CG |
| 5 | d6-6xHis-PUBTR- | 44 | AGG AGA TGA |
| | GA | | TCC GTT ATT |
| | | | CAT TAA TGG |
| | | | TGA |
| | | | TGA TGG TGG |
| | | | TGG GA |
| 6 | GA-pUBTR-XylA- | 55 | CAA TAA AAG |
| | 5′-F | | AGA AAG GTC |
| | | | GAC TCA TTT |
| | | | ACA |
| | | | TTT TAA CGA |
| | | | TAT CTA GAA |
| | | | AAT GTT C |
| 7 | GAPUBTR-R | 24 | GTC GAC CTT |
| | | | TCT CTT TTA |
| | | | TTG TCC |
| 8 | GA-pUBTR22b-R | 27 | CAT ATG TAA |
| | | | ATC GCT CCT |
| | | | TTT TAG GTG |

Constructs SE27a and SE27d were made from the DNA of SE27e to delete XylR/PxylA cassette (SE27a), or PyxiE promoter (SE27d) by PCR-amplifying with primers SEQ ID NO: 7 and SEQ ID NO: 4, or SEQ ID NO: 6 and SEQ ID NO: 8 (Table 3). The DNA of linearized constructs were gel-purified, and then re-circularized by site-directed mutagenesis in a KLD reaction (contains a blend of kinase, ligase and DpnI enzymes, NEB, Inc.), and transformed into B. subtilis cells. Plasmid DNA prepared from B. subtilis clones was verified by sequencing, and transformed into SE_{ΔΔΔ}. The plasmids from transformants were verified by PCR and sequencing of DNA preparations. All confirmed clones were stored in 20% glycerol at −80° C. Summary of ORF structure in SE27a, SE27d, and SE27e are represented in Table 4.

TABLE 4
Summary of ORF structure and Nucleic Acid
Sequences in SE27a, SE27d and SE27e

| Strain | |
|---|---|
| Gene/Expression | |
| Cassette | Nucleotide Sequence |
| SE27a | catatagaaataacgtcatttttat |
| “constitutive” | tttagttgctgaaaggtgcgttgaa |
| Phpall/PyxiE- | gtgttggtatgtatgtgttttaaag |
| RBSsod-yfhKSP- | tattgaaaacccttaaaattggttg |
| pro-LEKTI-D6 | cacagaaaaaccccatctgttaaag |
| SEQ ID NO: 10 | ttataagtgactaaacaaataacta |
| | aatagatgggggtttcttttaatat |
| | tatgtgtcctaatagtagcatttat |
| | tcagatgaaaaatcaagggttttag |
| | tggacaagacaaaaagtggaaaagt |
| | gagaccatggagagaaaagaaaatc |
| | gctaatgttgattactttgaacttc |
| | tgcatattcttgaatttaaaaaggc |
| | tgaaagagtaaaagattgtgctgaa |
| | atattagagtataaacaaaatcgtg |
| | aaacaggcgaaagaaagttgtatcg |
| | agtgtggttttgtaaatccaggctt |
| | tgtccaatgtgcaactggaggagag |
| | caatgaaacatggcattcagtcaca |
| | aaaggttgttgctgaagttattaaa |
| | caaaagccaacagttcgttggttgt |
| | ttctcacattaacagttaaaaatgt |
| | ttatgatggcgaagaattaaataag |
| | agtttgtcagatatggctcaaggat |
| | ttcgccgaatgatgcaatataaaaa |
| | aattaataaaaatcttgttggtttt |
| | atgcgtgcaacggaagtgacaataa |
| | ataataaagataattcttataatca |
| | gcacatgcatgtattggtatgtgtg |
| | gaaccaacttattttaagaatacag |
| | aaaactacgtgaatcaaaaacaatg |
| | gattcaattttggaaaaaggcaatg |
| | aaattagactatgatccaaatgtaa |
| | aagttcaaatgattcgaccgaaaaa |
| | taaatataaatcggatatacaatcg |
| | gcaattgacgaaactgcaaaatatc |
| | ctgtaaaggatacggattttatgac |
| | cgatgatgaagaaaagaatttgaaa |
| | cgtttgtctgatttggaggaaggtt |
| | tacaccgtaaaaggttaatctccta |
| | tggtggtttgttaaaagaaatacat |
| | aaaaaattaaaccttgatgacacag |
| | aagaaggcgatttgattcatacaga |
| | tgatgacgaaaaagccgatgaagat |
| | ggattttctattattgcaatgtgga |
| | attgggaacggaaaaattattttat |
| | taaagagtagttcaacaaacgggcc |
| | agtttgttgaagattagatgctata |
| | attgttattaaaaggattgaaggat |
| | gcttaggaagacgagttattatgac |
| | ctaggccgccaaaggccaaacatga |
| | tttggcctttttttcgttagacatc |
| | gtttccctttagcctttaattttag |
| | tatgatatgtaaatgatattgaata |
| | aaagctaggaagtgtcgtaatgagc |
| | acaaaacctttttacagagatacgt |
| | gggcggaaattgacttgtccgcgat |
| | aaaggaaaatgtcagcaatatgaaa |
| | aaacatatcggtgaacatgtccact |
| | tgatggcagttgtgaaagcaaacgc |
| | ctacgggcatggtgatgcagaaaca |
| | gcaaaggctgctcttgacgcaggtg |
| | cttcatgcttggccgtggccatttt |
| | ggatgaagcgatttcactgcgcaaa |
| | aagggattgaaggcgcctatattgg |
| | tgcttggcgcggttcccccggagta |
| | tgtggcaatcgctgctgagtatgac |
| | gtgaccttaacaggttattctgttg |
| | aatggcttcaggaggcagcccgcca |
| | cacgaaaaaaggttctcttcatttt |
| | catctgaaggtcgatacggggatga |
| | acagacttggtgtaaaaacagagga |
| | agaagttcagaacgtgatggcaatt |
| | cttgaccgcaaccctcgtttaaagt |
| | gcaaaggggtatttacccattttgc |
| | gacagcggatgaaaaagaaagaggc |
| | tatttcttaatgcagtttgagcgct |
| | ttaaagagctgattgctccgctgcc |
| | gttaaagaatctaatggtccactgc |
| | gcgaacagcgccgctggactccggc |
| | tgaaaaaaggcttttttaatgcagt |
| | cagattcggcatcggcatgtatg |
| SE27d “inducible” | catatagaaataacgtcatttttat |
| PhpaII/xyIR-PxyIA- | tttagttgctgaaaggtgcgttgaa |
| RBSsod-yfhKSP- | gtgttggtatgtatgtgttttaaag |
| pro--LEKTI-D6 | tattgaaaacccttaaaattggttg |
| SEQ ID NO: 11 | cacagaaaaaccccatctgttaaag |
| | ttataagtgactaaacaaataacta |
| | aatagatgggggtttcttttaatat |
| | tatgtgtcctaatagtagcatttat |
| | tcagatgaaaaatcaagggttttag |
| | tggacaagacaaaaagtggaaaagt |
| | gagaccatggagagaaaagaaaatc |
| | gctaatgttgattactttgaacttc |
| | tgcatattcttgaatttaaaaaggc |
| | tgaaagagtaaaagattgtgctgaa |
| | atattagagtataaacaaaatcgtg |
| | aaacaggcgaaagaaagttgtatcg |
| | agtgtggttttgtaaatccaggctt |
| | tgtccaatgtgcaactggaggagag |
| | caatgaaacatggcattcagtcaca |
| | aaaggttgttgctgaagttattaaa |
| | caaaagccaacagttcgttggttgt |
| | ttctcacattaacagttaaaaatgt |
| | ttatgatggcgaagaattaaataag |
| | agtttgtcagatatggctcaaggat |
| | ttcgccgaatgatgcaatataaaaa |
| | aattaataaaaatcttgttggtttt |
| | atgcgtgcaacggaagtgacaataa |
| | ataataaagataattcttataatca |
| | gcacatgcatgtattggtatgtgtg |
| | gaaccaacttattttaagaatacag |
| | aaaactacgtgaatcaaaaacaatg |
| | gattcaattttggaaaaaggcaatg |
| | aaattagactatgatccaaatgtaa |
| | aagttcaaatgattcgaccgaaaaa |
| | taaatataaatcggatatacaatcg |
| | gcaattgacgaaactgcaaaatatc |
| | ctgtaaaggatacggattttatgac |
| | cgatgatgaagaaaagaatttgaaa |
| | cgtttgtctgatttggaggaaggtt |
| | tacaccgtaaaaggttaatctccta |
| | tggtggtttgttaaaagaaatacat |
| | aaaaaattaaaccttgatgacacag |
| | aagaaggcgatttgattcatacaga |
| | tgatgacgaaaaagccgatgaagat |
| | ggattttctattattgcaatgtgga |
| | attgggaacggaaaaattattttat |
| | taaagagtagttcaacaaacgggcc |
| | agtttgttgaagattagatgctata |
| | attgttattaaaaggattgaaggat |
| | gcttaggaagacgagttattatgac |
| | ctaggccgccaaaggccaaacatga |
| | tttggcctttttttcgttagacatc |
| | gtttccctttagcctttaattttag |
| | tatgatatgtaaatgatattgaata |
| | aaagctaggaagtgtcgtaatgagc |
| | acaaaacctttttacagagatacgt |
| | gggcggaaattgacttgtccgcgat |
| | aaaggaaaatgtcagcaatatgaaa |
| | aaacatatcggtgaacatgtccact |
| | tgatggcagttgtgaaagcaaacgc |
| | ctacgggcatggtgatgcagaaaca |
| | gcaaaggctgctcttgacgcaggtg |
| | cttcatgcttggccgtggccatttt |
| | ggatgaagcgatttcactgcgcaaa |
| | aagggattgaaggcgcctatattgg |
| | tgcttggcgcggttcccccggagta |
| | tgtggcaatcgctgctgagtatgac |
| | gtgaccttaacaggttattctgttg |
| | aatggcttcaggaggcagcccgcca |
| | cacgaaaaaaggttctcttcatttt |
| | catctgaaggtcgatacggggatga |
| | acagacttggtgtaaaaacagagga |
| | agaagttcagaacgtgatggcaatt |
| | cttgaccgcaaccctcgtttaaagt |
| | gcaaaggggtatttacccattttgc |
| | gacagcggatgaaaaagaaagaggc |
| | tatttcttaatgcagtttgagcgct |
| | ttaaagagctgattgctccgctgcc |
| | gttaaagaatctaatggtccactgc |
| | gcgaacagcgccgctggactccggc |
| | tgaaaaaaggcttttttaatgcagt |
| | cagattcggcatcggcatgtatggc |
| | cttcgcccgtctgctgacatgtcgg |
| | acgagataccgtttcagctgcgtcc |
| | ggcatttaccctgcattcgacactg |
| | tcacatgtcaaactgatcagaaaag |
| | gcgagagcgtcagctacggagccga |
| | gtacacagcggaaaaagacacatgg |
| | atcgggacggtgcctgtaggctatg |
| | cggacggctggctccgaaaattgaa |
| | agggaccgacatccttgtgaaggga |
| | aaacgcctgaaaattgccggccgaa |
| | tttgcatggaccaatttatggtgga |
| | gctggatcaggaatatccgccgggc |
| | acaaaagtcacattaataggccggc |
| | agggggatgaatatatttccatgga |
| | tgagattgcaggaaggctcgaaacc |
| | attaactatgaggtggcctgtacaa |
| | taagttcccgtgttccccgtatgtt |
| | tttggaaaatgggagtataatggaa |
| | gtaagaaatcctttattgcaggtaa |
| | atataagcaattaaacgcgtaatct |
| | attattaatctgttcagcaatcggg |
| | cgcgattgctgaataaaagatacga |
| | gagacctctcttgtatcttttttat |
| | tttgagtggttttgtccgttacact |
| | agaaaaccgaaagacaataaaaatt |
| | ttattcttgctgagtctggctttcg |
| | gtaagctagacaaaacggacaaaat |
| | aaaaattggcaagggtttaaaggtg |
| | gagattttttgagtgatcttctcaa |
| | aaaatactacctgtcccttgctgat |
| | ttttaaacgagcacgagagcaaaac |
| | ccccctttgctgaggtggcagaggg |
| | caggtttttttgtttcttttttctc |
| | gtaaaaaaaagaaaggtcttaaagg |
| | ttttatggttttggtcggcactgcc |
| | gacagcctcgcagagcacacacttt |
| | atgaatataaagtatagtgtgttat |
| | actttacttggaagtggttgccgga |
| | aagagcgaaaatgcctcacatttgt |
| | gccacctaaaaaggagcgatttaca |
| | tatgcaataaaagagaaaggtcgac |
| | tcatttacattttaacgatatctag |
| | aaaatgttcaataacaatggaagac |
| | attcctaatgccggtgcattctttc |
| | ctaaggaagatggtaataattcata |
| | gctattgcctaattggggataaacc |
| | ctggacgacacttcacttctaattg |
| | aatttaaaaccataggatgtgattc |
| | aattatgctgtttcttaaaatgatg |
| | gcttgtggattgaatgtatttagaa |
| | tattagtaaggccaattcctaaata |
| | gaatccgaaattctgtaatgcattt |
| | aaggtgccgatatcattcagatggg |
| | cgaggtctatgatatcttgatagga |
| | cacttttttctctttagtctgaaga |
| | gattttaataaagccttctctgaag |
| | catacaattcccagcaacctcggtt |
| | tccgcaactgcatttaggaccatta |
| | aagtctattgtcatatgtcccattt |
| | ctccagagaatccgcttacccctct |
| | atataaatgattgttgataataaca |
| | ccgatccctatacctgtactgatac |
| | tagcataaataatgttattgtgatt |
| | ttttgcagcaccaaatactttttct |
| | ccatacgcgccagcatttgcctcat |
| | tttcaataaaaacaggcacattgaa |
| | cttctcttgtatgaaagattttaag |
| | tcaatatctctccagttggagttcg |
| | gagtgaaaacaattttttgattttt |
| | atcaattagtccaggcacgcatatg |
| | cctataccgataagcccgtacggag |
| | attgtggcatacgcgtaataaagtg |
| | atgaatcatatcaattaaaatgtct |
| | ttagttatttctggagaattcgatt |
| | ctaaatggtgatgttgatcaagaat |
| | gatcgttccttcaaggtctgttaaa |
| | atgccactaatataatccacaccaa |
| | catctattcctatggagtatcctgc |
| | ctttttattaaaaacaagcatgaca |
| | ggtcttcttccgccacttgattgtc |
| | cttgacctatttcaaatacaagatt |
| | ttctttcatcagcgtgtttacctgt |
| | gatgaaacagttgatttatttaatc |
| | cagtcatttcagataattttgctct |
| | tgaaataggtgaattcttaagtatt |
| | tcttttaataataacttttgattta |
| | cttttttgacaaaggtttgatctgc |
| | gatatccacttcatccactccattt |
| | gtataacctttaaattaagttaaaa |
| | ttttttgtgttcagtatgagattta |
| | gtacatagcgaatcttacctttatt |
| | atatctaatgtgttcatgaaaaact |
| | aaaaaaaatattgaaaatactgatg |
| | aggttatttaagattaaaataagtt |
| | agtttgtttgggcaacaaactaatg |
| | tgcaacttacttacaatatgacata |
| | aaatgcatctgtatttgaatttatt |
| | tttaaggaggaaataacatggctca |
| | atctcattctagttcagttaactat |
| | tttgtaagcgttaacaaagtggttt |
| | aattaaggtaccttaggaggatgat |
| | tatttatgaaaaagaaacaagtaat |
| | gctcgctttaacagctgccgcagga |
| | ctgggtttgacagcacttcattccg |
| | ctcccgcagcaaaagctgccgaatt |
| | cttagaaatttcatcaacatgtgat |
| | gcagaatctggtaaagcaacgagtt |
| | acgctgaattgtgctcagaataccg |
| | taagttagtcagaaacggaaagttg |
| | gcgtgtactcgtgagaacgacccta |
| | tccagggaccggacggcaaggtaca |
| | tggaaatacttgcagtatgtgcgag |
| | gtgtttttccaggcggaggaagaag |
| | aaaagaagaaaaaggagggcaaaag |
| | tcgaaattaatgaataatcggatca |
| | tctcctgcaggcagcacaattccaa |
| | gaaaaacacgatttagaacctaaaa |
| | agaacgaatttgaactaactcataa |
| | ccgagaggtaaaaaaagaacgaagt |
| | cgagatcagggaatgagtttataaa |
| | ataaaaaaagcacctgaaaaggtgt |
| | ctttttttgatggttttgaacttgt |
| | tctttcttatcttgata |
| SE27e “inducible” | catatagaaataacgtcatttttat |
| PhpaII/PyxiE/xylR- | tttagttgctgaaaggtgcgttgaa |
| PxylA-RBSsod- | gtgttggtatgtatgtgttttaaag |
| yfhKSP-pro-- | tattgaaaacccttaaaattggttg |
| LEKTI-D6 | cacagaaaaaccccatctgttaaag |
| SEQ ID NO: 12 | ttataagtgactaaacaaataacta |
| | aatagatgggggtttcttttaatat |
| | tatgtgtcctaatagtagcatttat |
| | tcagatgaaaaatcaagggttttag |
| | tggacaagacaaaaagtggaaaagt |
| | gagaccatggagagaaaagaaaatc |
| | gctaatgttgattactttgaacttc |
| | tgcatattcttgaatttaaaaaggc |
| | tgaaagagtaaaagattgtgctgaa |
| | atattagagtataaacaaaatcgtg |
| | aaacaggcgaaagaaagttgtatcg |
| | agtgtggttttgtaaatccaggctt |
| | tgtccaatgtgcaactggaggagag |
| | caatgaaacatggcattcagtcaca |
| | aaaggttgttgctgaagttattaaa |
| | caaaagccaacagttcgttggttgt |
| | ttctcacattaacagttaaaaatgt |
| | ttatgatggcgaagaattaaataag |
| | agtttgtcagatatggctcaaggat |
| | ttcgccgaatgatgcaatataaaaa |
| | aattaataaaaatcttgttggtttt |
| | atgcgtgcaacggaagtgacaataa |
| | ataataaagataattcttataatca |
| | gcacatgcatgtattggtatgtgtg |
| | gaaccaacttattttaagaatacag |
| | aaaactacgtgaatcaaaaacaatg |
| | gattcaattttggaaaaaggcaatg |
| | aaattagactatgatccaaatgtaa |
| | aagttcaaatgattcgaccgaaaaa |
| | taaatataaatcggatatacaatcg |
| | gcaattgacgaaactgcaaaatatc |
| | ctgtaaaggatacggattttatgac |
| | cgatgatgaagaaaagaatttgaaa |
| | cgtttgtctgatttggaggaaggtt |
| | tacaccgtaaaaggttaatctccta |
| | tggtggtttgttaaaagaaatacat |
| | aaaaaattaaaccttgatgacacag |
| | aagaaggcgatttgattcatacaga |
| | tgatgacgaaaaagccgatgaagat |
| | ggattttctattattgcaatgtgga |
| | attgggaacggaaaaattattttat |
| | taaagagtagttcaacaaacgggcc |
| | agtttgttgaagattagatgctata |
| | attgttattaaaaggattgaaggat |
| | gcttaggaagacgagttattatgac |
| | ctaggccgccaaaggccaaacatga |
| | tttggcctttttttcgttagacatc |
| | gtttccctttagcctttaattttag |
| | tatgatatgtaaatgatattgaata |
| | aaagctaggaagtgtcgtaatgagc |
| | acaaaacctttttacagagatacgt |
| | gggcggaaattgacttgtccgcgat |
| | aaaggaaaatgtcagcaatatgaaa |
| | aaacatatcggtgaacatgtccact |
| | tgatggcagttgtgaaagcaaacgc |
| | ctacgggcatggtgatgcagaaaca |
| | gcaaaggctgctcttgacgcaggtg |
| | cttcatgcttggccgtggccatttt |
| | ggatgaagcgatttcactgcgcaaa |
| | aagggattgaaggcgcctatattgg |
| | tgcttggcgcggttcccccggagta |
| | tgtggcaatcgctgctgagtatgac |
| | gtgaccttaacaggttattctgttg |
| | aatggcttcaggaggcagcccgcca |
| | cacgaaaaaaggttctcttcatttt |
| | catctgaaggtcgatacggggatga |
| | acagacttggtgtaaaaacagagga |
| | agaagttcagaacgtgatggcaatt |
| | cttgaccgcaaccctcgtttaaagt |
| | gcaaaggggtatttacccattttgc |
| | gacagcggatgaaaaagaaagaggc |
| | tatttcttaatgcagtttgagcgct |
| | ttaaagagctgattgctccgctgcc |
| | gttaaagaatctaatggtccactgc |
| | gcgaacagcgccgctggactccggc |
| | tgaaaaaaggcttttttaatgcagt |
| | cagattcggcatcggcatgtatggc |
| | cttcgcccgtctgctgacatgtcgg |
| | acgagataccgtttcagctgcgtcc |
| | ggcatttaccctgcattcgacactg |
| | tcacatgtcaaactgatcagaaaag |
| | gcgagagcgtcagctacggagccga |
| | gtacacagcggaaaaagacacatgg |
| | atcgggacggtgcctgtaggctatg |
| | cggacggctggctccgaaaattgaa |
| | agggaccgacatccttgtgaaggga |
| | aaacgcctgaaaattgccggccgaa |
| | tttgcatggaccaatttatggtgga |
| | gctggatcaggaatatccgccgggc |
| | acaaaagtcacattaataggccggc |
| | agggggatgaatatatttccatgga |
| | tgagattgcaggaaggctcgaaacc |
| | attaactatgaggtggcctgtacaa |
| | taagttcccgtgttccccgtatgtt |
| | tttggaaaatgggagtataatggaa |
| | gtaagaaatcctttattgcaggtaa |
| | atataagcaattaaacgcgtaatct |
| | attattaatctgttcagcaatcggg |
| | cgcgattgctgaataaaagatacga |
| | gagacctctcttgtatcttttttat |
| | tttgagtggttttgtccgttacact |
| | agaaaaccgaaagacaataaaaatt |
| | ttattcttgctgagtctggctttcg |
| | gtaagctagacaaaacggacaaaat |
| | aaaaattggcaagggtttaaaggtg |
| | gagattttttgagtgatcttctcaa |
| | aaaatactacctgtcccttgctgat |
| | ttttaaacgagcacgagagcaaaac |
| | ccccctttgctgaggtggcagaggg |
| | caggtttttttgtttcttttttctc |
| | gtaaaaaaaagaaaggtcttaaagg |
| | ttttatggttttggtcggcactgcc |
| | gacagcctcgcagagcacacacttt |
| | atgaatataaagtatagtgtgttat |
| | actttacttggaagtggttgccgga |
| | aagagcgaaaatgcctcacatttgt |
| | gccacctaaaaaggagcgatttaca |
| | tatgacgattacagaatcaactatc |
| | tgcgtgatcatttaattgaagcgcg |
| | cgaagccattgcggacggcgttgaa |
| | ttaatcggatacacatcatgggggc |
| | caattgaccttgtcagcgcatctac |
| | tgcggaaatgaaaaagcgctacggc |
| | ttcatttatgtagaccgggacaatg |
| | aaggaaatggaacatttaaccgcat |
| | caagaaaaaaagcttcaactggtat |
| | cagcaggttatcgccacaaacggag |
| | agagtctctgacaattttttgaaaa |
| | ctcatgcgcttcaattgacaataac |
| | gaaatgcaggcggacaataaaagag |
| | aaaggtcgactcatttacattttaa |
| | cgatatctagaaaatgttcaataac |
| | aatggaagacattcctaatgccggt |
| | gcattctttcctaaggaagatggta |
| | ataattcatagctattgcctaattg |
| | gggataaaccctggacgacacttca |
| | cttctaattgaatttaaaaccatag |
| | gatgtgattcaattatgctgtttct |
| | taaaatgatggcttgtggattgaat |
| | gtatttagaatattagtaaggccaa |
| | ttcctaaatagaatccgaaattctg |
| | taatgcatttaaggtgccgatatca |
| | ttcagatgggcgaggtctatgatat |
| | cttgataggacacttttttctcttt |
| | agtctgaagagattttaataaagcc |
| | ttctctgaagcatacaattcccagc |
| | aacctcggtttccgcaactgcattt |
| | aggaccattaaagtctattgtcata |
| | tgtcccatttctccagagaatccgc |
| | ttacccctctatataaatgattgtt |
| | gataataacaccgatccctatacct |
| | gtactgatactagcataaataatgt |
| | tattgtgattttttgcagcaccaaa |
| | tactttttctccatacgcgccagca |
| | tttgcctcattttcaataaaaacag |
| | gcacattgaacttctcttgtatgaa |
| | agattttaagtcaatatctctccag |
| | ttggagttcggagtgaaaacaattt |
| | tttgatttttatcaattagtccagg |
| | cacgcatatgcctataccgataagc |
| | ccgtacggagattgtggcatacgcg |
| | taataaagtgatgaatcatatcaat |
| | taaaatgtctttagttatttctgga |
| | gaattcgattctaaatggtgatgtt |
| | gatcaagaatgatcgttccttcaag |
| | gtctgttaaaatgccactaatataa |
| | tccacaccaacatctattcctatgg |
| | agtatcctgcctttttattaaaaac |
| | aagcatgacaggtcttcttccgcca |
| | cttgattgtccttgacctatttcaa |
| | atacaagattttctttcatcagcgt |
| | gtttacctgtgatgaaacagttgat |
| | ttatttaatccagtcatttcagata |
| | attttgctcttgaaataggtgaatt |
| | cttaagtatttcttttaataataac |
| | ttttgatttacttttttgacaaagg |
| | tttgatctgcgatatccacttcatc |
| | cactccatttgtataacctttaaat |
| | taagttaaaattttttgtgttcagt |
| | atgagatttagtacatagcgaatct |
| | tacctttattatatctaatgtgttc |
| | atgaaaaactaaaaaaaatattgaa |
| | aatactgatgaggttatttaagatt |
| | aaaataagttagtttgtttgggcaa |
| | caaactaatgtgcaacttacttaca |
| | atatgacataaaatgcatctgtatt |
| | tgaatttatttttaaggaggaaata |
| | acatggctcaatctcattctagttc |
| | agttaactattttgtaagcgttaac |
| | aaagtggtttaattaaggtacctta |
| | ggaggatgattatttatgaaaaaga |
| | aacaagtaatgctcgctttaacagc |
| | tgccgcaggactgggtttgacagca |
| | cttcattccgctcccgcagcaaaag |
| | ctgccgaattcttagaaatttcatc |
| | aacatgtgatgcagaatctggtaaa |
| | gcaacgagttacgctgaattgtgct |
| | cagaataccgtaagttagtcagaaa |
| | cggaaagttggcgtgtactcgtgag |
| | aacgaccctatccagggaccggacg |
| | gcaaggtacatggaaatacttgcag |
| | tatgtgcgaggtgtttttccaggcg |
| | gaggaagaagaaaagaagaaaaagg |
| | agggcaaaagtcgaaattaatgaat |
| | aacggatcatctcctgcaggcagca |
| | caattccaagaaaaacacgatttag |
| | aacctaaaaagaacgaatttgaact |
| | aactcataaccgagaggtaaaaaaa |
| | gaacgaagtcgagatcagggaatga |
| | gtttataaaataaaaaaagcacctg |
| | aaaaggtgtctttttttgatggttt |
| | tgaacttgttctttcttatcttgat |
| | a |

### Example 2

### Preparation of SE27a Lyophilized Powder

The SE27a strain was inoculated into two 500 mL cultures in 2 L baffled Erlenmeyer flasks. The cultures were grown at 30° C. and 250 rpm for 24 h. After incubation, the cultures were pooled, centrifuged at 10,000×g for 10 min at 4° C. and their supernatants were discarded. The pooled pellets were fully suspended in 80 mL of autoclaved cryoprotectant solution (120 g/L lactose, 25 g/L fructooligosaccharides and 1 g/L ascorbic acid) under magnetic stirring (Chen et al. 2019, Artif: Cells Nanomed. Biotechnol.). Two 100-μL aliquots of the homogenate were saved for plating and CFU assessment. The remainder of the bacterial suspension was placed into a UV-sterilized lyophilization flask and frozen in an ethanol-dry ice bath under constant manual spinning. The frozen paste was lyophilized for 48 h, scrapped under sterile conditions and the resulting powder was manually crushed in a stomacher bag. Three samples of the powder were taken, weighed and suspended in 10 mL of autoclaved 0.1×TSB (tryptic soy broth media) supplemented plus 12.5% Tween 80 (v/v). Following suspension, the homogenates were serially diluted and plated for CFU assessment.

This methodology yielded a total of 14.5 g of powder containing ˜34% of dried bacterial mass, as calculated by subtracting the mass of cryoprotectant from the total powder recovered. Prior to the lyophilization, the bacterial suspension in cryoprotectant contained a total of 9.1×10¹² CFU, of which 6.7×10¹² CFU were recovered after lyophilization. Thus, the process yields a 74% recover of the initial input, and the final bacterial density in the powder was 1.4×10¹² CFU/g of powder. Table 5 summarizes the components and percent (w/w).

TABLE 5
Cryoprotectants Used in Lyophilized SE27a.

| Component | % (w/w) |
|---|---|
| Lactose | 54.2 |
| FOS | 11.3 |
| Ascorbic Acid | 0.5 |
| SE27a | 34.0 |

### Example 3

### Methods

Growth and LEKTI expression is solution screening was carried out by growing the SE27a in liquid cultures of 30 g/L TSB media, artificial sweat media (ASM, 20.9 g/L MOPS, 2 g/L NaCl, 1 g/L yeast extract, 0.1 g/L Tween 80 and 0.65 mg/L cod liver fatty acid methyl esters), ASM plus 1% colloidal oatmeal (wt/v) or ASM plus 0.5% colloidal oatmeal extract (wt/v). The SE27a powder prepared in Example 2 was used to inoculate 50-mL liquid cultures in 250 mL flasks at a cell density of ˜10⁸ CFU/mL, and the cultures were grown at 30° C. and 250 rpm. In times, one-hundred μL cultures were taken, serially diluted in 10-fold steps using 1×PBS and plated onto 30 g/L TSB, 15 g/L NaCl and 15 g/L agar media for CFU assessment after growth at 37° C. for 24 h. Additionally, one-mL aliquots of the cultures were centrifuged at 12,000×g for 10 min and 4° C. for collection of the supernatant. These spent media samples were re-centrifuged a second time and subjected to active LEKTI determination using the assay described in below.

Active LEKTI concentration was tested using a KLK14 inhibition assay. To activate the pro-KLK14 form for the assay, this protein at 3.5 μM was activated using 0.3 μM thermolysis for 1 h at 37° C. in a solution containing 50 mM Tris, 10 mM CaCl₂, 150 mM NaCl, 0.05% (wt/v) Brij-35, pH 8.0. The reaction was quenched with 50 mM EDTA, and the samples were diluted 1:1 (v/v) in 50 mM Tris, 150 mM NaCl, 0.05% (wt/v) Brij-35 and 10% glycerol, pH 8.0. The aliquots were frozen at −80° C. and fully used in each assay to avoid freeze-thawing cycles. The amounts of active LEKTI in broth and extracts were assayed in black 96-well plates using a solution of 50 mM Tris, 150 mM NaCl and 0.05% (wt/v) Brij-35 (pH 8.0) containing 1.5 nM activated KLK14 and 30 μM Boc-VPR-AdMC. The reactions were performed in serial dilutions of media and extracts (10-0.08% of final well volume) and followed for 10 min in 1 min intervals at 23° C. in a plate reader (Ex/Em=380/460 nm). The inhibition of the KLK14-mediated cleavage of Boc-VPR-AdMC was determined as % reaction rate ±inhibitor. The concentration of active LEKTI in the samples was determined using a calibration curve of percent reaction rate in presence of 0.0-5.0 nM purified recombinant His-tagged LEKTI-d6.

### Evaluation of Added Nutrients in Solution

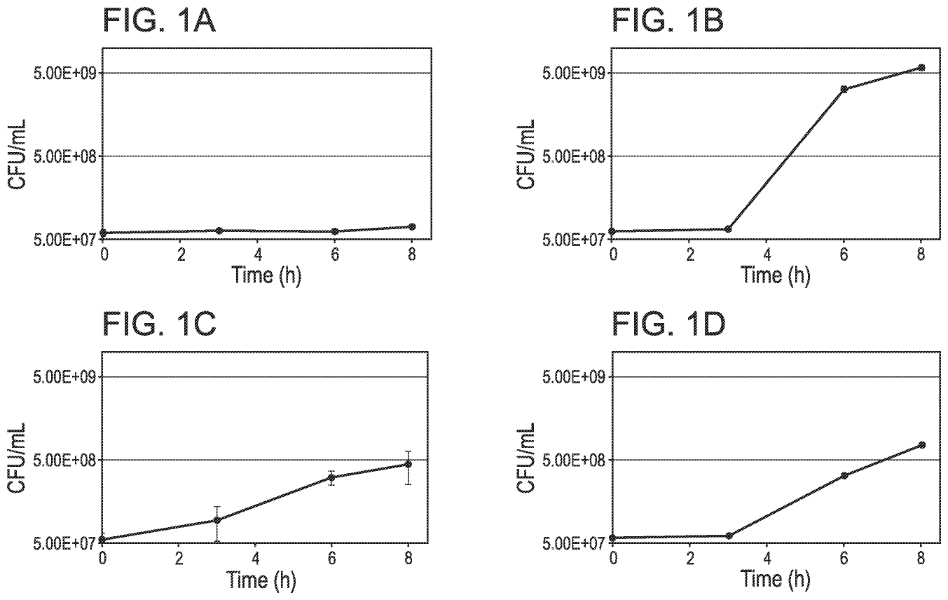

Liquid cultures of the S. epidermidis protein-expressing strain SE27a in defined volume in flasks in artificial sweat media (“ASM”—20.9 g/L MOPS, 2 g/L NaCl, 1 g/L yeast extract, 0.1 g/L Tween 80 and 0.65 mg/L cod liver fatty acid methyl esters (described by Oates and McBain, Biofouling 32(1): 25-33 (2016)) (FIG. 1A), tryptic soy broth (TSB) (FIG. 1B), ASM plus 1% colloidal oatmeal (CO) (FIG. 1C), and ASM plus 0.5% colloidal oatmeal extract (COE) (FIG. 1D). Samples were taken at selected time points that represented therapeutic timelines. The bacterial counts were measured (CFU/ml) using standard dilution protocol, and the “active protein concentration” was measured using in vitro KLK14 inhibition assay for ASM (FIG. 1E), TSB (FIG. 1F), ASM plus 1% colloidal oatmeal (CO) (FIG. 1G), and ASM plus 0.5% colloidal oatmeal extract (COE) (FIG. 1H).

The TSB control solutions grew (FIG. 1B) and expressed LEKTI (FIG. 1F) as expected. For the ASM alone (FIG. 1A), the solution contained an inoculum of 6.0×10⁷ CFU/ml of SE27a. At the 3-hour time point, the bacterial count was 6.3×10⁷ CFU/ml, representing a growth of 10%. The active LEKTI concentration was below the limit of detection (FIG. 1E). The solution containing ASM with 1% colloidal oatmeal had an inoculum of 5.6×10⁷ CFU/ml (FIG. 1C). After the 3-hour time point, the bacterial count was 9.6×10⁷ CFU/ml, or a growth of 71%. The active LEKTI concentration was 6.5 nM (FIG. 1G). For the solution of ASM with 0.5% colloidal oatmeal extract, the inoculum of SE27a was 5.8×10⁷ CFU/ml (FIG. 1D). At the 3-hour time point, the bacterial count was 6.2×10⁷ CFU/ml, representing a growth of 7%. The active LEKTI concentration was 215 nM (FIG. 1C).

The addition of colloidal oatmeal or colloidal oatmeal extract increases the LEKTI expression from below the limit of detection to considerable levels in 3 hours. These results indicate the addition of the oatmeal products will be useful to assure expression.

### Example 4

### Methods

Growth and LEKTI expression in different agar preparations were assessed by the following procedures. The SE27a strain powder was dissolved in 1×PBS to a cell density of 10¹¹ CFU/mL, and 100 μL at aliquots of these suspensions were spread onto the following agar plates (area of 78.5 cm²) containing 15 g/L bacteriological agar: 30 g/L TSB, ASM media, ASM plus 1% colloidal oatmeal wt/v and ASM plus 0.5% colloidal oatmeal extract wt/v. For each media, the plates contained approximately 10⁸ CFU/cm² and four plates were inoculated per experiment. The plates were incubated at 30° C. for 8 h and, at the end of this time, four plates for the same media condition were pooled and homogenized using a kitchen blender containing 200 mL of sterile H₂O. An aliquot of 100 μL of the homogenate was collected for plating and CFU assessment, while the remainder was centrifuged at 12,000×g for 20 min at 4° C. The pellet containing the agar was discarded, and the supernatant was re-centrifuged under the same conditions. The final liquid phase was frozen in a dry ice-ethanol bath and lyophilized for 48 h. The resulting dried material was dissolved in Milli-Q H₂O, acidified with 1% trifluoroacetic acid (TFA, v/v), injected into a preparative HPLC cartridge and fractionated at a flow rate of 10.9 mL/min in mobile phases containing (A) H₂O+0.1% TFA (v/v) and (B) acetonitrile (ACN)+0.1% TFA (v/v) according to the gradient described in Table 6.

TABLE 6
TFA and Acetonitrile gradients

| Time | % A (H₂O + | % B (ACN + |
|---|---|---|
| (min) | 0.1% TFA) | 0.1% TFA) |
| 0.0 | 95 | 5 |
| 0.3 | 70 | 30 |
| 20.0 | 70 | 30 |
| 20.2 | 20 | 80 |
| 30.2 | 20 | 80 |
| 30.3 | 95 | 5 |
| 33.3 | 95 | 5 |

The eluates fractions eluting between 20.2-30.2 min were collected, pooled and fully dried in a speedvac. The resulting pellet was dissolved in 100 μL of 0.5 M Tris, 75 mM NaCl and 0.03% (wt/v) Brij-35 (pH 8.0) and subjected to active LEKTI determination, as described above.

### Evaluation of Added Nutrients to Agar

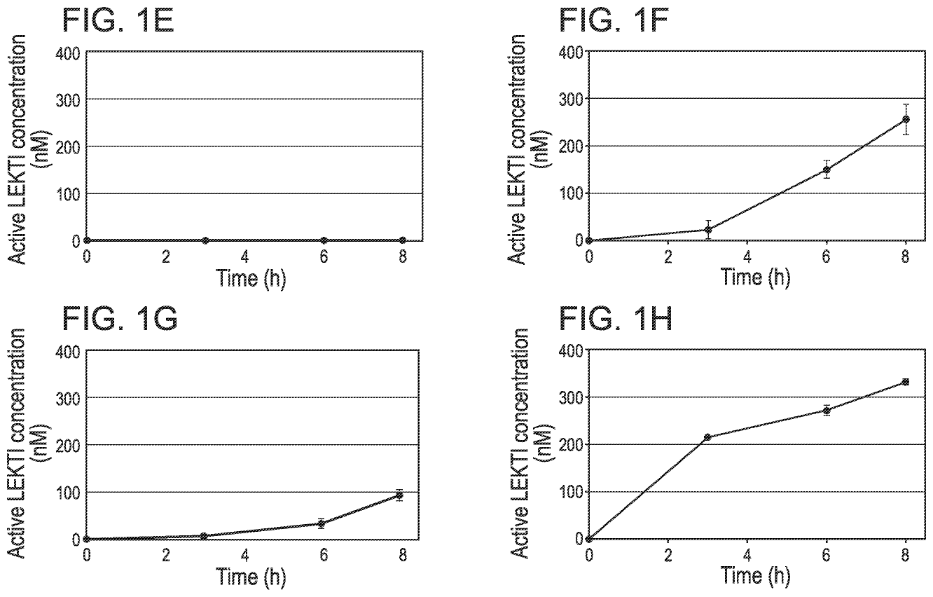

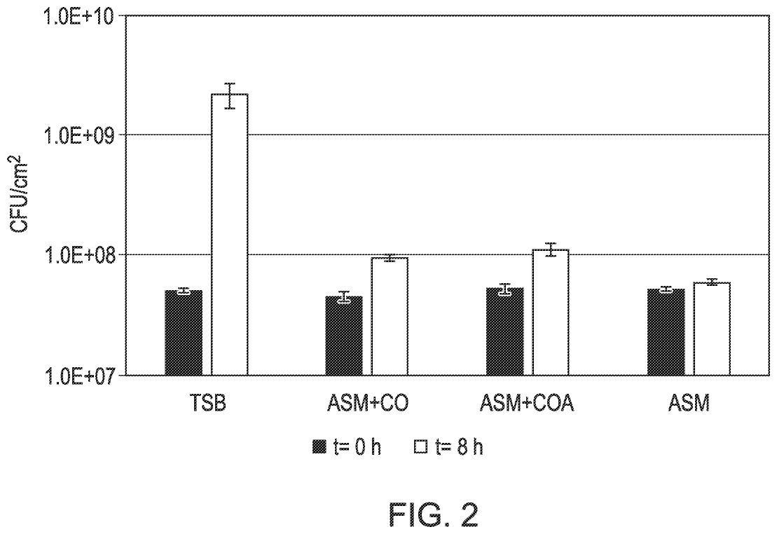

Colloidal oatmeal (CO) and colloidal oatmeal extract (COE) were mixed into ASM agar plates, bacteria applied and incubated at 30° C. for 8 hours. The amount of bacterial (CFU/cm²) and the active LEKTI-d6 protein expressed (KLK14 inhibition assay), measured at TO and at 8 hours (T8), are presented on the FIG. 2. FIG. 3 shows the active LEKTI-d6 levels for each condition.

The results showed there was little bacterial growth in the colloidal oatmeal or colloidal oatmeal extract conditions (FIG. 2). There was a high level of active LEKTI (FIG. 3) in the presence of colloidal oatmeal and colloidal oatmeal extract. There was 3 ng/cm² of LEKTI in the presence of colloidal oatmeal and 7 ng/cm² in the presence of colloidal oatmeal extract.

### Example 4

### Methods

Oleyl alcohol:cetyl alcohol 1:1±1% colloidal oatmeal (wt/wt) or 0.5% colloidal oatmeal extract (wt/wt) were prepared by incubating all components at 60° C. until the cetyl alcohol was completely melted. The mixtures were manually shaken until cooling and solidification. The final formulations were added to SE27a lyophilized powder in a proportion of 10:1 (wt/wt) using a stomacher bag. Briefly, the bacterial powder was placed in a stomacher bag and gently crushed using a glass rod. The formulation was then added and the bacterial powder plus formulation were homogenized with a glass rod using 25 up-and-down cycles of rolling motion externally to the bag. Aliquots of ˜100 mg of the formulations were spread onto ASM plates (area of 78.5 cm²) containing 15 g/L bacteriological agar, resulting in a bacterial density of ˜10⁸ CFU/cm². The plates were incubated at 30° C. for 8 h and, at the end of this time, four plates for the same media condition were pooled and homogenized using a kitchen blender containing 200 mL of sterile H₂O. An aliquot of 100 μL of the homogenate was collected for plating and CFU assessment, while the remainder was centrifuged at 12,000×g for 20 min at 4° C. The pellet containing the agar was discarded, and the supernatant was re-centrifuged under the same conditions. The final liquid phase was frozen in a dry ice-ethanol bath and lyophilized for 48 h. The resulting dried material was dissolved in Milli-Q H₂O, acidified with 1% trifluoroacetic acid (TFA, v/v), HPLC fractionated, dried, and assayed for active LEKTI levels as described above.

Alkaline extracts of colloidal oatmeal were prepared as follows. Colloidal oatmeal lots were obtained from commercial sources and were dissolved at 3% wt/v in aqueous 50 mM NaOH under magnetic stirring. The suspension was then incubated without stirring for 48 h at room temperature. At the end of this time, the suspension was neutralized with 50 mM HCl and the supernatant was carefully collected to avoid recovering of the decanted material. The supernatant was centrifuged twice at 12,000×g for 20 min at 4° C., frozen in a dry ice bath and lyophilized for 72 h. The dried material was scraped, weighed, and stored in a sealed stomacher bag at room temperature until use.

The colloidal oatmeal used as starting material and its corresponding alkaline extract were characterized using an iodine test to measure their starch content. Briefly, the colloidal oatmeal and the alkaline colloidal oatmeal extract were dissolved in 6 mL of MilliQ water at a concentration of 10 mg/mL. 0.2 mL of an ethanol solution of 10 mg/mL iodine was added to the mixtures. The color change to black was indicating the presence of starch in the suspensions. Colloidal oatmeal changed to black (positive for starch) while the alkaline colloidal oatmeal extract was clear (negative for starch).

### Evaluation of Added Nutrients to Formulation

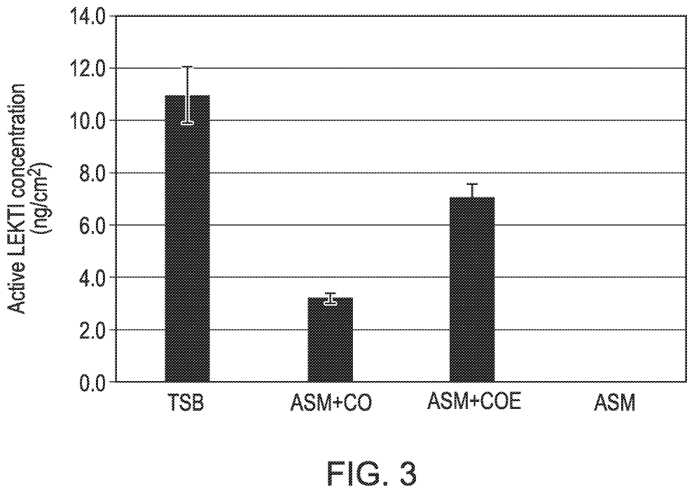

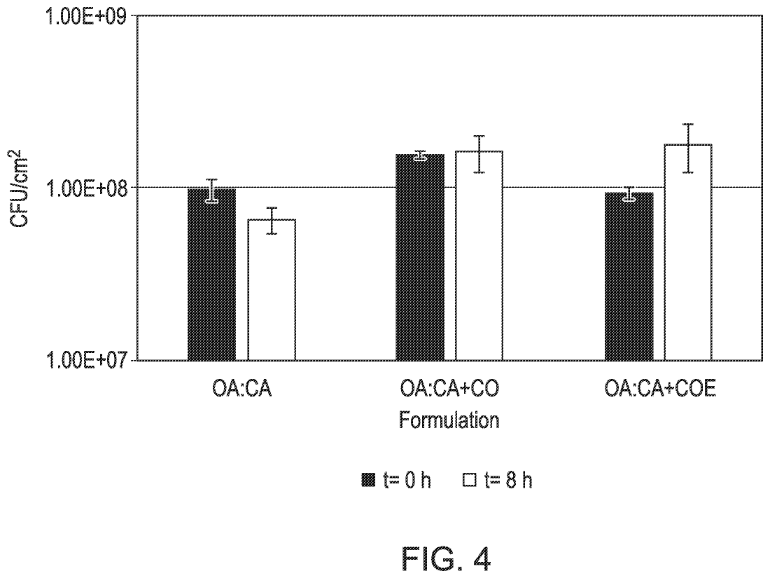

The SE27a was formulated in oleyl alcohol and cetyl alcohol 1:1 (OA:CA), OA:CA with colloidal oatmeal (CO) and OA:CA with colloidal oatmeal extract (COE), applied onto the ASM agar plate and then incubated at 30° C. for 8 hours. The bacterial growth under these conditions are presented on FIG. 4, and LEKTI expression is presented on FIG. 5.

SE27a in oleyl alcohol and cetyl alcohol 1:1 did not grow or express active LEKTI. There was no significant bacterial growth in the presence of colloidal oatmeal or colloidal oatmeal extract, as shown on FIG. 4. However, we observed that SE27a formulated in OA:CA with either 1% colloidal oatmeal (CO) or 0.5% colloidal oatmeal extract (COE) had the beneficial effect of expressing active LEKTI on a surface (ASM containing agar), as shown on FIG. 5.

### Example 5

### Methods

Placebo and 27a-containing formulations of oleyl alcohol:cetyl alcohol 1:1±1% colloidal oatmeal (wt/wt) were prepared as described under Example 4. The 27a powder was mixed with the formulation in a proportion of 10:1 (wt/wt) using a stomacher bag, as described under Example 4. Briefly, the bacterial powder was placed in a stomacher bag and gently crushed using a glass rod. Aliquots of ˜100-150 mg of the formulations were spread onto de-fattened and shaven pig skin from a local butcher (area of 64 cm²), resulting in a bacterial density of ˜10⁸ CFU/cm². The skin pieces containing placebo or 27a formulation were incubated at 30° C. for 0, 8 h and 24 h. At each time-point, two areas of 4 cm² were swabbed independently and subjected to two treatments. First, one swab was placed into a 15 mL Falcon tube containing 1 mL of a solution containing 3 g/L TSB powder and 1% Triton X-100 (wt/v). The tube was vortexed for 1 min, and its contents were serially diluted and plated for CFU assessment. The second swab was placed in a 15 mL Falcon tube containing a 1 mL of a solution of 50 mM Tris (pH=8.0), 150 mM NaCl and 0.05% Brij-35 (wt/v). The samples were vortexed for 1 min, centrifuged at 12,000×g for 1 min and their supernatant was collected. This fractions were centrifuged a second time under the same conditions, and the final supernatant was used for measurements of active LEKTI levels, as described under Example 3.

Bacterial Recovery and LEKTI Activity from Formulated 27a Applied on Pig Skin

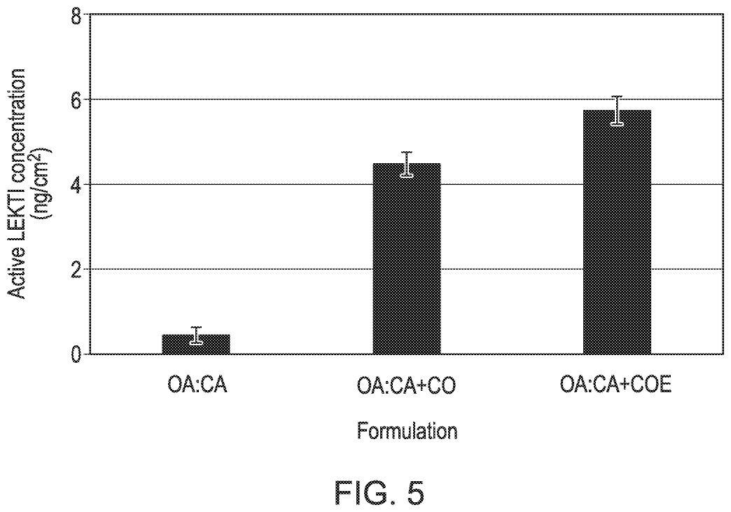

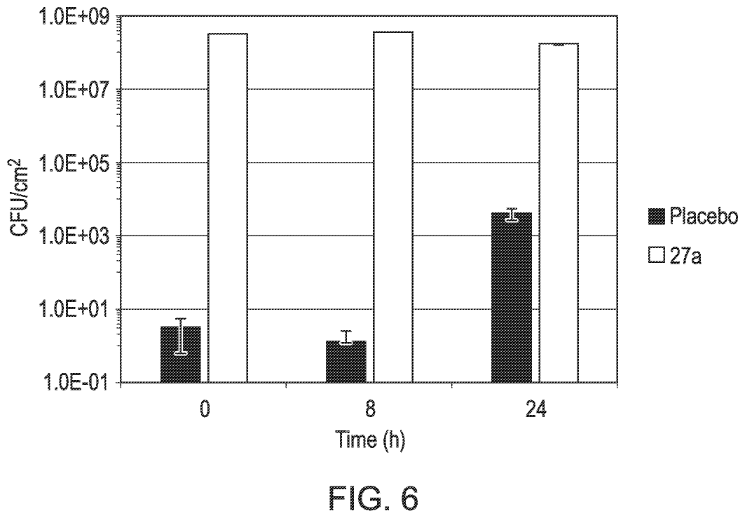

The SE27a was formulated in oleyl alcohol and cetyl alcohol 1:1 (OA:CA), with colloidal oatmeal (CO), applied onto the pig skin and then incubated at 30° C. for 8 hours. The bacterial content under these conditions is presented on FIG. 6, and LEKTI activity is presented on FIG. 7.

The formulated SE27a bacterial content was stable over 24 h on pig skin, as shown on FIG. 7. At T=0 h, no LEKTI activity was observed from samples applied to the pig skin. LEKTI activity sharply reached 60 ng/cm² in the swab samples from formulated 27a at T=8 h followed by a decrease to 34 ng/cm² at T=24 h, as shown in FIG. 7. Consistently with the observations from Example 5, the 27a bacteria formulated in OA:CA containing CO is able to produce active LEKTI on a pig skin surface without substantial expansion of their CFU content.

### Example 6

### Growth of Auxotroph and Natural Antimicrobial Production

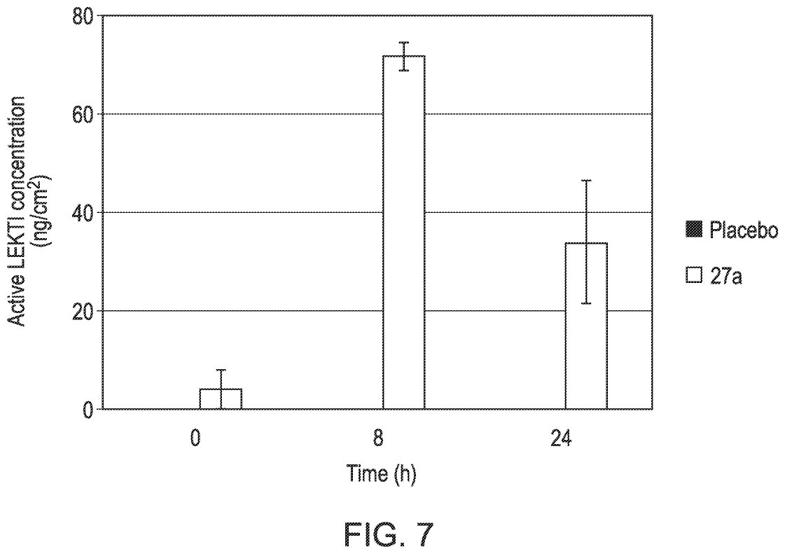

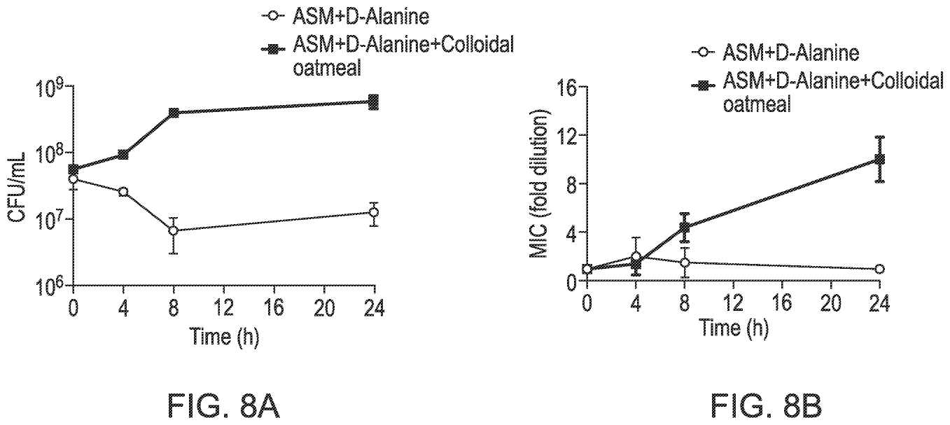

SE484 strain is a D-Alanine Staphylococcus epidermidis auxotrophic strain that produces and secretes antimicrobial compounds. In skin-like conditions, namely artificial sweat media (ASM), this strain's growth is impaired (FIG. 8A) and it is not able to produce and secrete antimicrobials (FIG. 8B). Upon supplementation with colloidal oatmeal, SE484 displays a 10-fold increase in CFU/mL within 8 h (FIG. 8A). Moreover, the spent broth of SE484 grown in media supplemented with colloidal oatmeal displays significant antibacterial activity against Bacillus subtilis168QC relative to plain ASM media (FIG. 8B).

### Example 7

### Bacterial Survival and Active LEKTI Production by Formulated SE351 on Ex Vivo Pig Skin

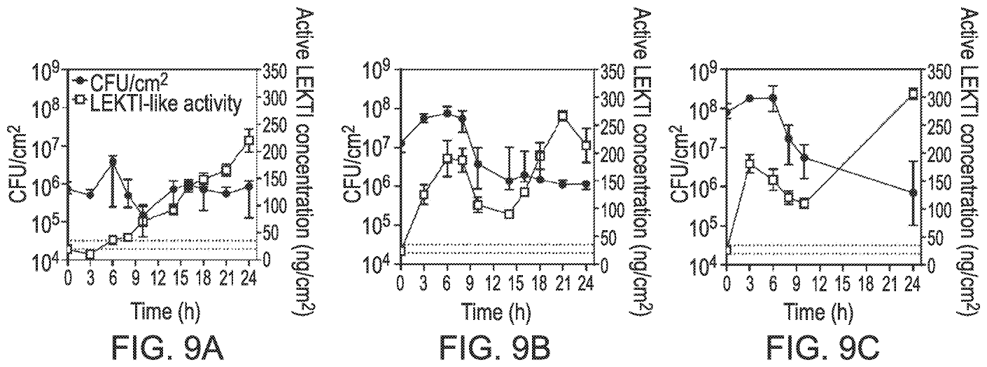

SE351 is a D-Alanine auxotrophic strain of S. epidermidis bearing a chromosomally integrated construct that expresses the domain 6 of the protease inhibitor LEKTI. When this strain is formulated in oleyl alcohol:cetyl alcohol 1:1 (w/w) supplemented with 1% colloidal oatmeal (w/w) and 2% D-Alanine (w/w) and applied on ex vivo pig skin at 106 (FIG. 9A), 10⁷ (FIG. 9B) and 10⁸ CFU/cm² (FIG. 9C) it displays a 10-fold increase in CFU/cm² within the first 6 h, and over 24 h, the bacterial population stabilizes around 10⁶ CFU/cm² in all cell densities (FIGS. 9A-9C). At 10⁶ CFU/cm², detectable active LEKTI levels are observed from 10-24 h, and gradually increase to a peak of ˜200 ng/cm² at 24 h (FIG. 9A).

At 10⁷ CFU/cm², active LEKTI levels display a first peak at 6-8 h, where levels reach 150 ng/cm² (FIG. 9B). Albeit those levels decrease to 100 ng/cm² between 10-14 h, they rise to a second peak of 250 ng/cm² within 20-24 h (FIG. 9B). A similar trend is observed for 10⁸ CFU/cm² (FIG. 9C), where LEKTI activity peaks firstly at 150 ng/cm² within 3 h, followed by a decrease and a second spike to 300 ng/cm² at 24 h. Despite the different kinetics of active LEKTI levels present in the three cell densities, SE351 formulated in an ointment containing colloidal oatmeal is capable of delivering substantial levels of active recombinant LEKTI on a skin surface.

### Example 8

### Preparation of Auxotrophic Strains SE351 and SE484 Lyophilized Powders

The SE351 and SE484 strains were inoculated into 500 mL TSB+2% D-Alanine (w/v) media placed in 2 L baffled Erlenmeyer flasks. The cultures were grown at 37° C. and 250 rpm for 6 h for SE351 and 16 h for SE484. After this incubation, the cultures were pooled, centrifuged at 10,000×g for 10 min at 4° C. and their supernatants were discarded. The pooled pellets were fully suspended in 80 mL of autoclaved cryoprotectant solution (120 g/L lactose, 25 g/L fructooligosaccharides and 1 g/L ascorbic acid) under magnetic stirring. Two 100-μL aliquots of the homogenate were saved for plating and CFU assessment. The remainder of the bacterial suspension was placed into a UV-sterilized lyophilization flask and frozen in an ethanol-dry ice bath under constant manual spinning. The frozen paste was lyophilized for 48 h, scrapped under sterile conditions and the resulting powder was manually crushed in a stomacher bag. Three samples of the powder were taken, weighed and suspended in 10 mL of autoclaved 0.1×TSB supplemented plus 12.5% Tween 80 (v/v). Following suspension, the homogenates were serially diluted and plated for CFU assessment. This methodology yielded a total of 8 g of powder per L of culture containing ˜34% of dried bacterial mass, as calculated by subtracting the mass of cryoprotectant from the total powder recovered. The CFU/g content varied from 1.0-2.5×10¹¹ CFU/g.

### Example 9

### Description of Growth of Auxotroph and Natural Antimicrobial Production

This screening was carried out by growing the SE484 in liquid cultures of artificial sweat media (ASM, 20.9 g/L MOPS, 2 g/L NaCl, 1 g/L yeast extract, 0.1 g/L Tween 80 and 0.65 mg/L cod liver fatty acid methyl esters) supplemented with 2% D-alanine (w/v) ±1% colloidal oatmeal (w/v). The SE484 powder prepared in EXAMPLE 8 was used to inoculate 50-mL liquid cultures in 250 mL flasks at a cell density of ˜10⁸ CFU/mL, and the cultures were grown at 30° C. and 250 rpm. In times, one-hundred μL cultures were taken, serially diluted in 10-fold steps using 1×PBS and plated onto 30 g/L TSB, 15 g/L NaCl and 15 g/L agar media for CFU assessment after growth at 37° C. for 24 h. Additionally, one-mL aliquots of the cultures were centrifuged at 12,000×g for 10 min and 4° C. for collection of the supernatant. These spent media samples were placed in 96-well microplates and serially diluted in TSB media using 2-fold steps. The serial dilutions of the spent broth were mixed 1:1 v/v of a TSB bacterial suspension of Bacillus subtilis 168 QC at 2×10⁵ CFU/mL, and the microplates were incubated at 37° C. for 16 h. The turbidity of the plates, which is an indicative of bacterial growth, were visually inspected. The wells in which the media was translucid were considered as containing high antibacterial activity, and the lowest concentration of spent broth, i.e., highest dilution, that resulted in such effect was considered the minimum inhibitory concentration (MIC) of the sample.

### Example 10

### Preparation of SE351 Formulated Products

Placebo formulations contained oleyl alcohol:cetyl alcohol 1:1 (w/w) supplemented with 1% colloidal oatmeal (w/w) and 2% D-Alanine (w/w) and were prepared by incubating all components at 60° C. until the cetyl alcohol was completely melted. The mixtures were manually shaken until cooling and solidification. The final placebo formulation was added to SE351 lyophilized powder (prepared as per EXAMPLE 8) in a proportion of 20:1 (w/w) using a stomacher bag. Briefly, the bacterial powder was placed in a stomacher bag and gently crushed using a glass rod. The formulation was then added and the bacterial powder plus formulation were homogenized with a glass rod using 25 up-and-down cycles of rolling motion externally to the bag. This process yielded a 10¹⁰ CFU/g formulation. Lower titer formulations at 10⁹ and 10⁸ CFU/g were obtained by mixing aliquots of the 10¹⁰ CFU/g formulation with fresh placebo at 1:10 and 1:100 w/w proportions, respectively. The mixing was carried in stomacher bags with the aid of a glass rod, as described above.

### Example 11

### Bacterial Survival and Active LEKTI Production by Formulated SE351 on Ex Vivo Pig Skin

Freshly prepared, shaven and de-fattened pig skin was obtained from a local butcher and used within 24 h of preparation. For each experiment, a large piece of pig skin was divided in three 36 cm² rectangular samples, and each part was treated with placebo or SE351 formulations at ˜10 mg/cm². Briefly, the formulation was added to the skin, and pressed with a glove-covered finger. The applied formulation was then rubbed in horizontal motions for 15 times to allow the formation of a homogeneous, thin film of ointment on top of the skin. The weight of the pig skin plus retained formulation was determined. Application of the SE351 ointment yielded bacterial densities of 10⁶-10⁸ CFU/cm². The skin pieces were then incubated at 30° C., and in times, areas of 4 cm² were rubbed with a polyester tip flocked swab using 10 strokes in vertical and 10 strokes in horizontal directions. For each treatment (placebo and SE351), two sets of swabs were collected per time point: the sets used for CFU/cm² determination were immediately processed after collection, whereas those collected for protein extract preparation were placed into Precellys homogenization tubes and stored at −20° C. until use.

For CFU/cm² determination, the swabs collected from formulation-treated pig skin were immediately transferred to a 15 mL Falcon tube, where they were treated with 0.5 mL of a sterile solution of 3 g/L TSB and 1% Triton X-100 (w/v). The tubes were vortexed for 1 min and aliquots of the homogenates were serially diluted in PBS using 10-fold step increments. The diluted samples were plated onto TSB-agar plates supplemented with 15 g/L NaCl and grown for 24 h at 37° C. The CFU content was assessed and normalized by swabbed area.

Protein extracts prepared for active LEKTI measurements were prepared from the second set of swabs from the pig skin through an extensive cleanup procedure to remove the interference of the matrix in the biochemical measurements. Briefly, the swabs in Precellys homogenization tubes were treated with 1 mL of a homogenization solution (90% methanol v/v and 0.5% formic acid v/v). The tubes were capped, and homogenized in a bead beater at 2,500 rpm for 30 s. The swabs were removed from the tubes, and the homogenate centrifuged at 12,000×g for 5 min and 20° C. The supernatant of the mixture was collected and speedvac'ed for 3-4 h at room temperature until complete dryness. The dry pellets were suspended in 250 μL of homogenization solution, sonicated for 1 min and treated with 750 μL of an 8 M guanidine-HCl solution. After mixing by inversion, 250 μL of pure hexane was added to each tube followed by five rounds of manual inversion. The tubes were centrifuged at 12,000×g for 5 min and 20° C., and the upper phase and interface containing the residual formulation components were discarded. The lower phase was transferred to 3 kDa Amicon concentrators and washed six times with 500 μL of 50 mM Tris pH 8.0, following the manufacturer's specifications. The final retentate was dislodged from the filter by a reverse spin with 250 μL of aqueous 50% acetonitrile (v/v), 0.025% Brij-35 (w/v) and 0.25% formic acid (v/v). The suspended retentates were speedvac'ed for 24 h at room temperature, and the final pellets were reconstituted in 50 μL of 0.25 M Tris pH 8.0. These samples were used for and titration of KLK14 inhibitory activity and total trypsin-like activity, as described in EXAMPLE 2.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A composition comprising one or more viable microorganisms and a first mixture, wherein the one or more microorganisms produces a therapeutic agent, wherein the first mixture comprises one or more disaccharides, one or more oligosaccharides, and one or more antioxidants, and wherein the composition has viability of at least 1×10¹⁰ CFU/g.

2. The composition of claim 1, further comprising a second mixture of one or more fatty alcohols or alcohol.

3. The composition of claim 1, wherein the one or more disaccharides is selected from the group consisting of lactose, trehalose, sucrose, maltose, and cellobiose; and/or
wherein the one or more oligosaccharides is selected from the group consisting of fructo-oligosaccharides (FOS), galactooligosaccharides (GOS), mannan oligosaccharides (MOS), isomalto-oligosaccharides, xylo-oligosaccharides, galacto-oligosaccharides, and raffinose; and/or
wherein the one or more antioxidants is selected from the group consisting of ascorbic acid, vitamin A, vitamin E, a-carotene, lycopene, lutein, zeaxanthin, and water soluble derivatives of lipophilic antioxidants.

4. (canceled)

5. (canceled)

6. The composition of claim 3, wherein the water soluble derivatives of lipophilic antioxidants are alpha-tocopherol phosphate or alpha-tocopherol polyethylene glycol ester.

7. The composition of claim 3, wherein the one or more disaccharides is lactose, the one or more oligosaccharides is fructo-oligosaccharides (FOS), and the one or more antioxidants is ascorbic acid.

8. The composition of claim 2, wherein the one or more fatty alcohols is selected from the group consisting of saturated fatty alcohol and unsaturated fatty alcohol.

9. The composition of claim 8, wherein the saturated fatty or alcohol acid is cetyl alcohol and the unsaturated fatty alcohol is oleyl alcohol.

10. The composition of claim 9, wherein the second mixture of oleyl alcohol and cetyl alcohol ranges from about 25% oleyl alcohol/75% cetyl alcohol to about 50% oleyl alcohol/50% cetyl alcohol (w/w), and/or
wherein the second mixture comprises oleyl alcohol and cetyl alcohol at about a 1:1 ratio.

11. (canceled)

12. The composition of claim 1, wherein the one or more disaccharides is about 50% (w/w) to about 55% (w/w), the one or more oligosaccharides is about 10% (w/w) to about 15% (w/w), the one or more antioxidants is about 0.1% (w/w) to about 2% (w/w), and the one or more microorganism is about 30% (w/w) to about 35% (w/w).

13. The composition of claim 1, wherein the composition of the microorganisms and the first mixture is about a 10:1 ratio with the second mixture comprising the one or more fatty alcohols.

14. The composition of claim 13, wherein the second mixture further comprises colloidal oatmeal and/or alkaline oatmeal extract, and/or wherein the second mixture comprises colloidal oatmeal at about 1% (wt/wt) and the alkaline oatmeal extract at about 0.5% (wt/wt) when in combination with the fatty alcohols.

15. (canceled)

16. The composition of claim 1, wherein the microorganism is selected from the group consisting of Bifidobacterium, Brevibacterium, Propionibacterium, Lactococcus, Streptococcus, Staphylococcus, Lactobacillus, Enterococcus, Pediococcus, Leuconostoc, Oenococcus, or Corynebacterium and mixtures thereof.

17. (canceled)

18. The composition of claim 1, wherein the therapeutic agent is produced naturally by the microorganism, or wherein the microorganism is genetically engineered to produce the therapeutic agent.

19. (canceled)

20. The composition of claim 1, wherein the therapeutic agent is selected from a polypeptide, a small molecule, and a metabolite.

21. The composition of claim 1, wherein the therapeutic agent is selected from the group consisting of one or more LEKTI protein domains, filaggrins, interferons, enkephalins, interleukins, and antimicrobial agents,
wherein the one or more antimicrobial agents is selected from the group consisting of chitinase, a glucanase, or a peptidoglycan hydrolase.

22. The composition of claim 21, wherein the one or more LEKTI protein domains is LEKTI-d6.

23. (canceled)

24. The composition of claim 1, wherein the microorganism is attenuated by auxotrophy, and wherein the microorganism comprises a deletion of one or more of alr1, alr2, and dat genes.

25. (canceled)

26. (canceled)

27. The composition of claim 1, wherein the composition is administered to a mammal's skin.

28. (canceled)

29. The composition of claim 1, wherein the expression of the therapeutic agent is at least 2-fold, at least 3-fold, at least 4-fold, at least 5-fold, at least 6-fold, at least 7-fold, at least 8-fold, at least 9-fold, at least 10-fold, at least 11-fold, at least 12-fold, or at least 13-fold when compared to a composition without the second mixture after 8 hours.

30. (canceled)

31. (canceled)

32. The composition of claim 1, wherein the composition has viability of at least 2.5×10¹⁰ CFU/g, at least 5×10¹⁰ CFU/g, at least 1×10¹¹ CFU/g, at least 2.5×10¹¹ CFU/g, at least 5×10¹¹ CFU/g, at least 1×10¹² CFU/g, or at least 2.5×10¹² CFU/g; and/or
wherein the composition has viability of about 1×10¹⁰ CFU/g to about 1×10¹¹ CFU/g, about 5×10¹⁰ CFU/g to about 5×10¹¹ CFU/g, about 1×10¹¹ CFU/g to about 1×10¹² CFU/g, or about 5×10¹¹ CFU/g to about 5×10¹² CFU/g.

33. (canceled)

34. The composition of claim 21, wherein the antimicrobial activity is at least 1.5-fold, at least 2-fold, at least 3-fold, at least 4-fold, or at least 5-fold when compared to a composition without the second mixture after 8 hours; and/or
wherein the antimicrobial activity is at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, or at least 12-fold when compared to a composition without the second mixture after 24 hours.

35. (canceled)

36. The composition of claim 21, wherein the LEKTI protein domains activity is at least 1.5-fold, at least 2-fold, at least 3-fold, at least 4-fold, or at least 5-fold when compared to a composition without the second mixture after 8 hours; and/or
wherein the LEKTI protein domains activity is at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, or at least 12-fold when compared to a composition without the second mixture after 24 hours.

37. (canceled)

38. A pharmaceutical composition comprising the composition of claim 1, and a pharmaceutically acceptable carrier, and
wherein the pharmaceutically acceptable carrier is selected from the group consisting of an aqueous solution, an emulsion, a cream, a lotion, a gel, or an ointment.

39. (canceled)

40. A method for preparing the composition of claim 1, comprising:
(a) combining the one or more microorganisms and the first mixture, wherein the first mixture comprises the one or more disaccharides, the one or more oligosaccharides, and the one or more antioxidants;
(b) lyophilizing the resulting composition of step (a); and
(c) combining the second mixture and the resulting lyophilized composition of step (b), wherein the second mixture comprises the one or more fatty alcohols.

41. A method of treating a disease, disorder, or condition in a subject, the method comprising administering to the subject the composition of claim 1, wherein the disease, disorder, or condition is a skin disease or disorder, inflammatory disease, cancer, or associated with pain, and
wherein the skin disease or disorder is Netherton Syndrome, psoriasis, acne, atopic dermatitis, allergic contact dermatitis, epidermolytic hyperkeratosis, seborrheic dermatitis, eczema, dry skin, allergy, rashes, UV-irritated skin, detergent irritated skin, thinning skin, bullous pemphigoid, pemphigus vulgaris, impetigo, vitiligio, baldness, and/or hirsutism, or
wherein the cancer is selected from the group consisting of malignant melanoma, colon cancer, breast cancer, lung cancer, ovarian cancer, gastric cancer, oral tongue squamous cell carcinoma, squamous cell cancer, prostate cancer, pancreatic cancer, liver cancer, kidney cancer, bladder cancer, cervical cancer, endometrial cancer, gallbladder cancer, brain cancer and oral cancer.

42.-45. (canceled)

46. A method of increasing expression of a therapeutic agent by a microorganism, comprising preparing the composition of claim 1, comprising:
(a) combining the one or more microorganisms and the first mixture, wherein the first mixture comprises the one or more disaccharides, the one or more oligosaccharides, and the one or more antioxidants;
(b) lyophilizing the resulting composition of step (a); and
(c) combining the second mixture and the resulting lyophilized composition of step (b), wherein the second mixture comprises the one or more fatty alcohols.


SEQUENCE LISTING (119 sequences)

SEQ ID NO: 1 - DNA, 26 bp, synthetic construct
  misc_feature 1..26: note = Description of Artificial Sequence: Synthetic primer
  source 1..26: mol_type = other DNA
cataaataatcatcctcctaaggtac

SEQ ID NO: 2 - DNA, 33 bp, synthetic construct
  misc_feature 1..33: note = Description of Artificial Sequence: Synthetic primer
  source 1..33: mol_type = other DNA
aatgaataacggatcatctcctgcaggcagcac

SEQ ID NO: 3 - DNA, 42 bp, synthetic construct
  misc_feature 1..42: note = Description of Artificial Sequence: Synthetic primer
  source 1..42: mol_type = other DNA
ggagatgatccgttattcattaatttcgacttttgccctcct

SEQ ID NO: 4 - DNA, 47 bp, synthetic construct
  misc_feature 1..47: note = Description of Artificial Sequence: Synthetic primer
  source 1..47: mol_type = other DNA
taccttaggaggatgattatttatgaaaaagaaacaagtaatgctcg

SEQ ID NO: 5 - DNA, 44 bp, synthetic construct
  misc_feature 1..44: note = Description of Artificial Sequence: Synthetic primer
  source 1..44: mol_type = other DNA
aggagatgatccgttattcattaatggtgatgatggtggtggga

SEQ ID NO: 6 - DNA, 55 bp, synthetic construct
  misc_feature 1..55: note = Description of Artificial Sequence: Synthetic primer
  source 1..55: mol_type = other DNA
caataaaagagaaaggtcgactcatttacattttaacgatatctagaaaatgttc

SEQ ID NO: 7 - DNA, 24 bp, synthetic construct
  misc_feature 1..24: note = Description of Artificial Sequence: Synthetic primer
  source 1..24: mol_type = other DNA
gtcgacctttctcttttattgtcc

SEQ ID NO: 8 - DNA, 27 bp, synthetic construct
  misc_feature 1..27: note = Description of Artificial Sequence: Synthetic primer
  source 1..27: mol_type = other DNA
catatgtaaatcgctcctttttaggtg

SEQ ID NO: 9 - skipped

SEQ ID NO: 10 - DNA, 2048 bp, synthetic construct
  misc_feature 1..2048: note = Description of Artificial Sequence: Synthetic polynucleotide
  source 1..2048: mol_type = other DNA
catatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattatgacctaggccgccaaaggccaaacatgatttggcctttttttcgttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatg

SEQ ID NO: 11 - DNA, 5117 bp, synthetic construct
  misc_feature 1..5117: note = Description of Artificial Sequence: Synthetic polynucleotide
  source 1..5117: mol_type = other DNA
catatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattatgacctaggccgccaaaggccaaacatgatttggcctttttttcgttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaaacgcgtaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgcaataaaagagaaaggtcgactcatttacattttaacgatatctagaaaatgttcaataacaatggaagacattcctaatgccggtgcattctttcctaaggaagatggtaataattcatagctattgcctaattggggataaaccctggacgacacttcacttctaattgaatttaaaaccataggatgtgattcaattatgctgtttcttaaaatgatggcttgtggattgaatgtatttagaatattagtaaggccaattcctaaatagaatccgaaattctgtaatgcatttaaggtgccgatatcattcagatgggcgaggtctatgatatcttgataggacacttttttctctttagtctgaagagattttaataaagccttctctgaagcatacaattcccagcaacctcggtttccgcaactgcatttaggaccattaaagtctattgtcatatgtcccatttctccagagaatccgcttacccctctatataaatgattgttgataataacaccgatccctatacctgtactgatactagcataaataatgttattgtgattttttgcagcaccaaatactttttctccatacgcgccagcatttgcctcattttcaataaaaacaggcacattgaacttctcttgtatgaaagattttaagtcaatatctctccagttggagttcggagtgaaaacaattttttgatttttatcaattagtccaggcacgcatatgcctataccgataagcccgtacggagattgtggcatacgcgtaataaagtgatgaatcatatcaattaaaatgtctttagttatttctggagaattcgattctaaatggtgatgttgatcaagaatgatcgttccttcaaggtctgttaaaatgccactaatataatccacaccaacatctattcctatggagtatcctgcctttttattaaaaacaagcatgacaggtcttcttccgccacttgattgtccttgacctatttcaaatacaagattttctttcatcagcgtgtttacctgtgatgaaacagttgatttatttaatccagtcatttcagataattttgctcttgaaataggtgaattcttaagtatttcttttaataataacttttgatttacttttttgacaaaggtttgatctgcgatatccacttcatccactccatttgtataacctttaaattaagttaaaattttttgtgttcagtatgagatttagtacatagcgaatcttacctttattatatctaatgtgttcatgaaaaactaaaaaaaatattgaaaatactgatgaggttatttaagattaaaataagttagtttgtttgggcaacaaactaatgtgcaacttacttacaatatgacataaaatgcatctgtatttgaatttatttttaaggaggaaataacatggctcaatctcattctagttcagttaactattttgtaagcgttaacaaagtggtttaattaaggtaccttaggaggatgattatttatgaaaaagaaacaagtaatgctcgctttaacagctgccgcaggactgggtttgacagcacttcattccgctcccgcagcaaaagctgccgaattcttagaaatttcatcaacatgtgatgcagaatctggtaaagcaacgagttacgctgaattgtgctcagaataccgtaagttagtcagaaacggaaagttggcgtgtactcgtgagaacgaccctatccagggaccggacggcaaggtacatggaaatacttgcagtatgtgcgaggtgtttttccaggcggaggaagaagaaaagaagaaaaaggagggcaaaagtcgaaattaatgaataatcggatcatctcctgcaggcagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgata

SEQ ID NO: 12 - DNA, 5426 bp, synthetic construct
  misc_feature 1..5426: note = Description of Artificial Sequence: Synthetic polynucleotide
  source 1..5426: mol_type = other DNA
catatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattatgacctaggccgccaaaggccaaacatgatttggcctttttttcgttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaaacgcgtaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgacgattacagaatcaactatctgcgtgatcatttaattgaagcgcgcgaagccattgcggacggcgttgaattaatcggatacacatcatgggggccaattgaccttgtcagcgcatctactgcggaaatgaaaaagcgctacggcttcatttatgtagaccgggacaatgaaggaaatggaacatttaaccgcatcaagaaaaaaagcttcaactggtatcagcaggttatcgccacaaacggagagagtctctgacaattttttgaaaactcatgcgcttcaattgacaataacgaaatgcaggcggacaataaaagagaaaggtcgactcatttacattttaacgatatctagaaaatgttcaataacaatggaagacattcctaatgccggtgcattctttcctaaggaagatggtaataattcatagctattgcctaattggggataaaccctggacgacacttcacttctaattgaatttaaaaccataggatgtgattcaattatgctgtttcttaaaatgatggcttgtggattgaatgtatttagaatattagtaaggccaattcctaaatagaatccgaaattctgtaatgcatttaaggtgccgatatcattcagatgggcgaggtctatgatatcttgataggacacttttttctctttagtctgaagagattttaataaagccttctctgaagcatacaattcccagcaacctcggtttccgcaactgcatttaggaccattaaagtctattgtcatatgtcccatttctccagagaatccgcttacccctctatataaatgattgttgataataacaccgatccctatacctgtactgatactagcataaataatgttattgtgattttttgcagcaccaaatactttttctccatacgcgccagcatttgcctcattttcaataaaaacaggcacattgaacttctcttgtatgaaagattttaagtcaatatctctccagttggagttcggagtgaaaacaattttttgatttttatcaattagtccaggcacgcatatgcctataccgataagcccgtacggagattgtggcatacgcgtaataaagtgatgaatcatatcaattaaaatgtctttagttatttctggagaattcgattctaaatggtgatgttgatcaagaatgatcgttccttcaaggtctgttaaaatgccactaatataatccacaccaacatctattcctatggagtatcctgcctttttattaaaaacaagcatgacaggtcttcttccgccacttgattgtccttgacctatttcaaatacaagattttctttcatcagcgtgtttacctgtgatgaaacagttgatttatttaatccagtcatttcagataattttgctcttgaaataggtgaattcttaagtatttcttttaataataacttttgatttacttttttgacaaaggtttgatctgcgatatccacttcatccactccatttgtataacctttaaattaagttaaaattttttgtgttcagtatgagatttagtacatagcgaatcttacctttattatatctaatgtgttcatgaaaaactaaaaaaaatattgaaaatactgatgaggttatttaagattaaaataagttagtttgtttgggcaacaaactaatgtgcaacttacttacaatatgacataaaatgcatctgtatttgaatttatttttaaggaggaaataacatggctcaatctcattctagttcagttaactattttgtaagcgttaacaaagtggtttaattaaggtaccttaggaggatgattatttatgaaaaagaaacaagtaatgctcgctttaacagctgccgcaggactgggtttgacagcacttcattccgctcccgcagcaaaagctgccgaattcttagaaatttcatcaacatgtgatgcagaatctggtaaagcaacgagttacgctgaattgtgctcagaataccgtaagttagtcagaaacggaaagttggcgtgtactcgtgagaacgaccctatccagggaccggacggcaaggtacatggaaatacttgcagtatgtgcgaggtgtttttccaggcggaggaagaagaaaagaagaaaaaggagggcaaaagtcgaaattaatgaataacggatcatctcctgcaggcagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgata

SEQ ID NO: 13-102 - skipped (90 entries)

SEQ ID NO: 103 - protein, 1064 aa, unidentified
  REGION 1..1064: note = Description of Unknown: LEKTI protein sequence
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 104 - protein, 55 aa, unidentified
  REGION 1..55: note = Description of Unknown: LEKTI Domain 1 sequence
KNEDQEMCHEFQAFMKNGKLFCPQDKKFFQSLDGIMFINKCATCKMILEKEAKSQ

SEQ ID NO: 105 - protein, 63 aa, unidentified
  REGION 1..63: note = Description of Unknown: LEKTI Domain 2 sequence
APTELNCDDFKKGERDGDFICPDYYEAVCGTDGKTYDNRCALCAENAKTGSQIGVKSEGECKS

SEQ ID NO: 106 - protein, 62 aa, unidentified
  REGION 1..62: note = Description of Unknown: LEKTI Domain 3 sequence
NPEQDVCSAFRPFVRDGRLGCTRENDPVLGPDGKTHGNKCAMCAELFLKEAENAKREGETRI

SEQ ID NO: 107 - protein, 67 aa, unidentified
  REGION 1..67: note = Description of Unknown: LEKTI Domain 4 sequence
NAEKDFCKEYEKQVRNGRLFCTRESDPVRGPDGRMHGNKCALCAEIFKQRFSEENSKTDQNLGKAEE

SEQ ID NO: 108 - protein, 62 aa, unidentified
  REGION 1..62: note = Description of Unknown: LEKTI Domain 5 sequence
REIVKLCSQYQNQAKNGILFCTRENDPIRGPDGKMHGNLCSMCQAYFQAENEEKKKAEARAR

SEQ ID NO: 109 - protein, 68 aa, unidentified
  REGION 1..68: note = Description of Unknown: LEKTI Domain 6 sequence
ESGKATSYAELCSEYRKLVRNGKLACTRENDPIQGPDGKVHGNTCSMCEVFFQAEEEEKKKKEGKSRN

SEQ ID NO: 110 - protein, 59 aa, unidentified
  REGION 1..59: note = Description of Unknown: LEKTI Domain 7 sequence
ASFEELCSEYRKSRKNGRLFCTRENDPIQGPDGKMHGNTCSMCEAFFQQEERARAKAKR

SEQ ID NO: 111 - protein, 62 aa, unidentified
  REGION 1..62: note = Description of Unknown: LEKTI Domain 8 sequence
EAAKEICSEFRDQVRNGTLICTREHNPVRGPDGKMHGNKCAMCASVFKLEEEEKKNDKEEKG

SEQ ID NO: 112 - protein, 62 aa, unidentified
  REGION 1..62: note = Description of Unknown: LEKTI Domain 9 sequence
EAVQELCSEYRHYVRNGRLPCTRENDPIEGLDGKIHGNTCSMCEAFFQQEAKEKERAEPRAK

SEQ ID NO: 113 - protein, 63 aa, unidentified
  REGION 1..63: note = Description of Unknown: LEKTI Domain 10 sequence
EAEKETCDEFRRLLQNGKLFCTRENDPVRGPDGKTHGNKCAMCKAVFQKENEERKRKEEEDQR

SEQ ID NO: 114 - protein, 57 aa, unidentified
  REGION 1..57: note = Description of Unknown: LEKTI Domain 11 sequence
GNTQDECAEYREQMKNGRLSCTRESDPVRDADGKSYNNQCTMCKAKLEREAERKNEY

SEQ ID NO: 115 - protein, 63 aa, unidentified
  REGION 1..63: note = Description of Unknown: LEKTI Domain 12 sequence
ESGKDTCDEFRSQMKNGKLICTRESDPVRGPDGKTHGNKCTMCKEKLEREAAEKKKKEDEDRS

SEQ ID NO: 116 - protein, 63 aa, unidentified
  REGION 1..63: note = Description of Unknown: LEKTI Domain 13 sequence
NDKEDLCREFRSMQRNGKLICTRENNPVRGPYGKMHINKCAMCQSIFDREANERKKKDEEKSS

SEQ ID NO: 117 - protein, 62 aa, unidentified
  REGION 1..62: note = Description of Unknown: LEKTI Domain 14 sequence
NNAKDECSEFRNYIRNNELICPRENDPVHGADGKFYTNKCYMCRAVFLTEALERAKLQEKPS

SEQ ID NO: 118 - protein, 62 aa, unidentified
  REGION 1..62: note = Description of Unknown: LEKTI Domain 15 sequence
SLDSEMCKDYRVLPRIGYLCPKDLKPVCGDDGQTYNNPCMLCHENLIRQTNTHIRSTGKCEE

SEQ ID NO: 119 - DNA, 3192 bp, synthetic construct
  misc_feature 1..3192: note = Description of Artificial Sequence: Synthetic polynucleotide
  source 1..3192: mol_type = other DNA
atgaagatagccacagtgtcagtgcttctgcccttggctctttgcctcatacaagatgctgccagtaagaatgaagatcaggaaatgtgccatgaatttcaggcatttatgaaaaatggaaaactgttctgtccccaggataagaaattttttcaaagtcttgatggaataatgttcatcaataaatgtgccacgtgcaaaatgatactggaaaaagaagcaaaatcacagaagagggccaggcatttagcaagagctcccaaggctactgccccaacagagctgaattgtgatgattttaaaaaaggagaaagagatggggattttatctgtcctgattattatgaagctgtttgtggcacagatgggaaaacatatgacaacagatgtgcactgtgtgctgagaatgcgaaaaccgggtcccaaattggtgtaaaaagtgaaggggaatgtaagagcagtaatccagagcaggatgtatgcagtgcttttcggccctttgttagagatggaagacttggatgcacaagggaaaatgatcctgttcttggtcctgatgggaagacgcatggcaataagtgtgcaatgtgtgctgagctgtttttaaaagaagctgaaaatgccaagcgagagggtgaaactagaattcgacgaaatgctgaaaaggatttttgcaaggaatatgaaaaacaagtgagaaatggaaggcttttttgtacacgggagagtgatccagtccgtggccctgacggcaggatgcatggcaacaaatgtgccctgtgtgctgaaattttcaagcagcgtttttcagaggaaaacagtaaaacagatcaaaatttgggaaaagctgaagaaaaaactaaagttaaaagagaaattgtgaaactctgcagtcaatatcaaaatcaggcaaagaatggaatacttttctgtaccagagaaaatgaccctattcgtggtccagatgggaaaatgcatggcaacttgtgttccatgtgtcaagcctacttccaagcagaaaatgaagaaaagaaaaaggctgaagcacgagctagaaacaaaagagaatctggaaaagcaacctcatatgcagagctttgcagtgaatatcgaaagcttgtgaggaacggaaaacttgcttgcaccagagagaacgatcctatccagggcccagatgggaaagtgcatggcaacacctgctccatgtgtgaggtcttcttccaagcagaagaagaagaaaagaaaaagaaggaaggtaaatcaagaaacaaaagacaatctaagagtacagcttcctttgaggagttgtgtagtgaataccgcaaatccaggaaaaacggacggcttttttgcaccagagagaatgaccccatccagggcccagatggaaaaatgcatggcaacacctgctccatgtgtgaggccttctttcaacaagaagaaagagcaagagcaaaggctaaaagagaagctgcaaaggaaatctgcagtgaatttcgggaccaagtgaggaatggaacacttatatgcaccagggagcataatcctgtccgtggcccagatggcaaaatgcatggaaacaagtgtgccatgtgtgccagtgtgttcaaacttgaagaagaagagaagaaaaatgataaagaagaaaaagggaaagtcgaggctgaaaaagttaagagagaagcagttcaggagctgtgcagtgaatatcgtcattatgtgaggaatggacgactcccctgtaccagagagaatgatcctattgagggtctagatgggaaaatccacggcaacacctgctccatgtgtgaagccttcttccagcaagaagcaaaagaaaaagaaagagctgaacccagagcaaaagtcaaaagagaagctgaaaaggagacatgcgatgaatttcggagacttttgcaaaatggaaaacttttctgcacaagagaaaatgatcctgtgcgtggcccagatggcaagacccatggcaacaagtgtgccatgtgtaaggcagtcttccagaaagaaaatgaggaaagaaagaggaaagaagaggaagatcagagaaatgctgcaggacatggttccagtggtggtggaggaggaaacactcaggacgaatgtgctgagtatcgggaacaaatgaaaaatggaagactcagctgtactcgggagagtgatcctgtacgtgatgctgatggcaaatcgtacaacaatcagtgtaccatgtgtaaagcaaaattggaaagagaagcagagagaaaaaatgagtattctcgctccagatcaaatgggactggatcagaatcagggaaggatacatgtgatgagtttagaagccaaatgaaaaatggaaaactcatctgcactcgagaaagtgaccctgtccggggtccagatggcaagacacatggcaataagtgtactatgtgtaaggaaaaactggaaagggaagcagctgaaaaaaaaaagaaagaggatgaagacaggagcaatacaggagaaaggagcaatacaggagaaaggagcaatgacaaagaggatctgtgtcgtgaatttcgaagcatgcagagaaatggaaagcttatctgcaccagagaaaataaccctgttcgaggcccatatggcaagatgcacatcaataaatgtgctatgtgtcagagcatctttgatcgagaagctaatgaaagaaaaaagaaagatgaagagaaatcaagtagcaagccctcaaataatgcaaaggatgagtgcagtgaatttcgaaactatataaggaacaatgaactcatctgccctagagagaatgacccagtgcacggtgctgatggaaagttctatacaaacaagtgctacatgtgcagagctgtctttctaacagaagctttggaaagggcaaagcttcaagaaaagccatcccatgttagagcttctcaagaggaagacagcccagactctttcagttctctggattctgagatgtgcaaagactaccgagtattgcccaggataggttatctttgtccaaaggatttaaagcctgtctgtggtgacgatggccaaacctacaacaatccttgcatgctctgtcatgaaaacctgatacgccaaacaaatacacacatccgcagtacagggaagtgtgaggagagcagcaccccaggaaccaccgcagccagcatgcccccgtctgacgaa